(19) United States
(12) Patent Application Publication
(10) Pub. No.: US 2011/0124888 A1
(43) Pub. Date: May 26, 2011

(54) STYRYLBENZOFURAN DERIVATIVES AS INHIBITORS FOR BETA-AMYLOID FIBRIL FORMATION AND PREPARATION METHOD THEREOF

(75) Inventors: Dong Jin Kim, Seoul (KR); Kyung Ho Yoo, Seoul (KR); Ji Hun Byun, Seoul (KR); YoungSoo Kim, Yongin-si (KR); Hye Yun Kim, Seoul (KR); Gwan Sun Lee, Seoul (KR); Maeng Sup Kim, Seoul (KR); Young Gil Ahn, Seongnam-si (KR); Ji Hoon Lee, Seoul (KR); Myoung-Hwan Lee, Seoul (KR); Hana Hwang, Anyang-si (KR); Jiyeon Ryu, Seoul (KR)

(73) Assignees: Korea Institute of Science and Technology, Seoul (KR); HANMI HOLDINGS CO., LTD, Seoul (KR)

(21) Appl. No.: 12/997,397

(22) PCT Filed: Jun. 12, 2009
(86) PCT No.: PCT/KR2009/003165

§ 371 Date: Dec. 10, 2010

(30) Foreign Application Priority Data
Jun. 12, 2008 (KR) 10-2008-0055307

(51) Int. Cl. C07D 307/81; C07D 307/80

(52) U.S. Cl. 549/469

(57) ABSTRACT

The present invention relates to a novel compound which efficiently inhibits the formation of beta-amyloid fibrils in the brain to be useful for preventing or treating a degenerative brain disease, a method for preparing same, and a pharmaceutical composition comprising same as an active ingredient.

### FIELD OF THE INVENTION

The present invention relates to a novel compound for inhibiting the formation of senile plaques caused by the accumulation of beta-amyloid, a method for preparing same, and a pharmaceutical composition for preventing or treating a degenerative brain disease comprising same as an active ingredient.

### BACKGROUND OF THE INVENTION

An ever increasing number of the elderly population gets afflicted by degenerative brain diseases such as senile dementia, cerebral apoplexy, and Parkinson's disease, which has become a major social problem, particularly because no effective drugs or methods for preventing and treating such diseases are currently available.

Commercially available drugs for Alzheimer's disease such as Tackrin® (Warner-Lambert), Aricept® (Eisai) and ExcelIon® (Novartis) function to suppress acetylcholine esterases to increase the concentration of acetylcholine which is a cholinergic neurotransmitter, thereby temporarily enhancing the cognitive capability, rather than blocking the accumulation of beta-amyloid protein itself.

Alzheimer's disease is a particularly serious form of the senile dementia, and it has been found that a major cause of the disease is the neurotoxicity arising from the accumulation of beta-amyloid proteins in the brain. Specifically, beta-amyloid protein precursors (APP) are converted to beta-amyloid 42 (Aβ42) monomers by the actions of β- and γ-secretases, and the Aβ42 monomers tend to gradually form oligomers, protofibrils, fibrils, and plaques, which are deposited in the brain. Accordingly, there has existed a need for developing a therapeutic agent which is capable of selectively recognizing beta-amyloid and blocking the fibril formation therefrom.

As potential beta-amyloid, β- and γ-secretases inhibitors, metal chelates, beta-amyloid vaccines, statin-based drugs, and nonsteroidal anti-inflammatory drugs have been studied. In the study of beta-amyloid vaccine AN-1792(Elan), when transgenic mice over-expressing beta-amyloid are administered with AN-1792, there have been generated antibodies capable of inhibiting beta-amyloid protein accumulation and clearing amyloid plagues formed in the brain of the transgenic mice: In case of young mice, the formation of senile plaques was inhibited, while in case of old mice, the progress of the senile plaque formation was delayed (Schenk, D. et al. Nature, 400, 173 (1999)). The above study shows that agents which inhibit the formation of olygomers or senile plaques are useful for preventing or treating a degenerative brain disease such as Alzheimer' dementia.

Pharmaceutical agents designed to deal with beta-amyloid are generally divided into two classes, therapeutic agents and diagnostic molecular imaging agents depending on the target, mode of action and pharmacokinetics.

Beta-amyloid fibril comprises 90% of beta-amyloid 40 (Aβ40) and 10% of beta-amyloid 42 (Aβ42) (Bitan, G et al., Proc. Natl. Sci. USA, 100, 330., (2003), and Jan, A. et al., J. Biol. Chem., 283, 28176, (2008)), and beta-amyloid 42 exhibits a strong neurotoxicity to induce apotosis of brain cells. Therefore, beta-amyloid 42 is the major target of a therapeutic drug, while beta-amyloid 40 is that of a diagnostic agent. In terms of the mode of action, a therapeutic agent acts on soluble monomers and lower oligomers having an α-helix structure to inhibit the generation of insoluble oligomers which are 5 times more neurotoxic than fibrils, whereas a diagnostic agent having a β-plated sheet type-structure exhibits a high binding affinity to insoluble oligomers. In terms of the pharmacokinetics, a therapeutic agent for degenerative brain diseases has different biodynamics from that of a diagnostic agent. A diagnostic agent is required to be capable of quickly penetrating into the brain blood so that the diagonosis of a patient can be performed within the half life of the radioisotope used therein. Fast clearance (CL) of the diagnostic agent remaining after the diagnostic procedure is also required, so that the exact amount of diagnostic agent bonded to the target can be analyzed (Mathis, C. A. et al., Curr. Pharm. Design, 10, 1469, (2004)). In case of a therapeutic agent, however, an overall high concentration over a required time period (area under the concentration versus time curve, AUC) is required together with a high absorbability.

There have been disclosed a number of compounds or extracts useful for the inhibition of the beta-amyloid fibril formation, and examples thereof include: detergents such as hexadecyl-N-methyl piperidinium (HMPBr); anti-cancer antibiotic agents such as doxorubicin; benzofuran derivatives such as SKF-74652 (Howlett, D. R. et al., Biochem. J. 343, 419 (1999)); human acetylcholine secretases (HuAchE) such as propidium (Bartolini, M. et al., Biochem. Pharmacol., 65, 407 (2003)]); a Ginko biloba extract designated LB-152 (Lin, S. et al., Bioorg. Med. Chem. Lett., 14, 1173 (2004)); a curry extract named curcumin (Yang, F. et al., J. Biol. Chem., 280, 5892 (2005)); and nordihydro guaiaretic acid (NDGA) (Ono, K. et al., Biochem. Biophys. Res. Commun., 330, 111 (2005)).

Among the aforementioned compounds and extracts, however, the compounds of a pseudo-peptide type suffer from the problems of low bioavailability and poor stability due to their high molecular weights, and the anti-cancer antibiotic agents cause adverse side effects when administered over a long period of time. Further, it has been reported that the reported compounds and extracts have difficulties in meeting the requirement that a brain disease therapeutic agent must be able to effectively penetrate through the brain blood barrier (BBB).

The present inventors have therefore endeavored to develop a novel compound which is free from the above problems and is effective for preventing or treating a disease associated with the accumulation of beta-amyloid fibrils in the brain, and have found that a specific styrylbenzofuran derivative exhibits a high inhibitory effect on beta-amyloid 42 and enhanced brain blood barrier penetrating capability, without causing undesirable side effects.

### SUMMARY OF THE INVENTION

Accordingly, it is a primary object of the present invention to provide a novel compound or a pharmaceutically acceptable salt thereof which efficiently inhibits the formation of beta-amyloid fibrils.

It is another object of the present invention to provide a method for preparing said compound.

It is another object of the present invention to provide a phamarceutical composition for inhibiting the formation of beta-amyloid fibrils, which comprises said compound or a pharmaceutically acceptable salt thereof as an active ingredient.

It is a further object of the present invention to provide a phamarceutical composition for preventing or treating a degenerative brain disease, which comprises said compound or a pharmaceutically acceptable salt thereof as an active ingredient.

In accordance with one aspect of the present invention, there is provided a compound of formula (I) or a pharmaceutically acceptable salt thereof:

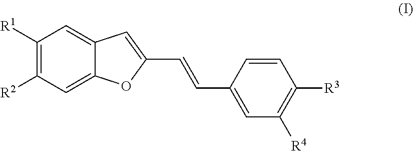

wherein

R¹ and R² are each independently H, OH, halogen, C₁-C₃ alkoxy, C₁-C₃ alkyl, substituted poly(C₁-C₃ alkoxy) having one or more halogen or hydroxyl groups, or substituted pyranyl(C₁-C₃ alkoxy) having one or more C₁-C₃ alkyl groups;

R³ is NH₂, C₁-C₃ alkylamino, C₁-C₃ dialkylamino, or C₁-C₃ alkoxy; and

R⁴ is H or C₁-C₃ alkoxy.

In accordance with other aspect of the present invention, there is provided a preparation method of the compound of formula (I).

In accordance with further aspect of the present invention, there is provided a phamarceutical composition comprising the compound of formula (I), or the pharmaceutically acceptable salt thereof as an active ingredient for inhibiting the formation of beta-amyloid fibrils.

In accordance with another aspect of the present invention, there is provided a pharmaceutical composition comprising the compound of formula (I), or the pharmaceutically acceptable salt thereof as an active ingredient for the prevention and treatment of a degenerative brain disease.

### BRIEF DESCRIPTION OF THE DRAWINGS

The above and other objects and features of the present invention will become apparent from the following description of the invention, when taken in conjunction with the accompanying drawings which respectively show:

FIG. 1: photographs of the hippocampus tissues of the transgenic mice stained by the compound of Example 9 as well as by tramiprosate (comparative compound).

FIG. 2: photographs of the cortex tissues of the transgenic mice stained by the compound of Example 9 as well as by tramiprosate (comparative compound).

### DETAILED DESCRIPTION OF THE INVENTION

As used herein, the term “alkyl” refers to a straight or branched chain saturated C₁-C₃ hydrocarbon radical. Examples of “alkyl” as used herein include, but are not limited to, methyl, ethyl, n-propyl, and isopropyl.

As used herein, the term “alkoxy” refers to the group —ORₐ, where Rₐ is alkyl as defined above. Exemplary alkoxy groups useful in the present invention include, but are not limited to, methoxy, ethoxy, n-propoxy, and isopropoxy.

As used herein, the term “halogen” refers to fluorine (F), chlorine (Cl), bromine (Br), or iodine (I).

Preferably, the compound of formula (I) according to the present invention may be a compound, wherein

R¹ and R² are each independently H, OH, halogen, OCH₃, CH₃, (OCH₂CH₂)₂F, (OCH₂CH₂)₃F, or dimethylpyranylmethoxy;

R³ is NH₂, NHCH₃, N(CH₃)₂, or OCH₃; and

R⁴ is H or OCH₃.

Examples of preferred styrylbenzofuran derivatives of formula (I) according to the present invention are:
(1) 2-(4-dimethylaminostyryl)benzofuran;
(2) 5-methoxy-2-(4-dimethylaminostyryl)benzofuran;
(3) 5-hydroxy-2-(4-dimethylaminostyryl)benzofuran;
(4) 5-methyl-2-(4-dimethylaminostyryl)benzofuran;
(5) 5-fluoro-2-(4-dimethylaminostyryl)benzofuran;
(6) 5-chloro-2-(4-dimethylaminostyryl)benzofuran;
(7) 5-bromo-2-(4-dimethylaminostyryl)benzofuran;
(8) 5-iodo-2-(4-dimethylaminostyryl)benzofuran;
(9) 6-methoxy-2-(4-dimethylaminostyryl)benzofuran;
(10) 6-hydroxy-2-(4-dimethylaminostyryl)benzofuran;
(11) 6-methyl-2-(4-dimethylaminostyryl)benzofuran;
(12) 6-fluoro-2-(4-dimethylaminostyryl)benzofuran;
(13) 6-chloro-2-(4-dimethylaminostyryl)benzofuran;
(14) 6-bromo-2-(4-dimethylaminostyryl)benzofuran;
(15) 6-iodo-2-(4-dimethylaminostyryl)benzofuran;
(16) 5-methoxy-2-(4-aminostyryl)benzofuran;
(17) 5-methoxy-2-(4-methylaminostyryl)benzofuran;
(18) 5-hydroxy-2-(4-aminostyryl)benzofuran hydrochloride;
(19) 5-hydroxy-2-(4-methylaminostyryl)benzofuran hydrochloride;
(20) 6-methoxy-2-(4-aminostyryl)benzofuran;
(21) 6-methoxy-2-(4-methylaminostyryl)benzofuran;
(22) 5-methoxy-2-(3-Methoxy-4-dimethylaminostyryl)benzofuran;
(23) 6-methoxy-2-(3-methoxy-4-dimethylaminostyryl)benzofuran;
(24) 2-(4-aminostyryl)benzofuran trifluoroacetate;
(25) 2-(4-methylaminostyryl)benzofuran trifluoroacetate;
(26) 2-(4-diethylaminostyryl)benzofuran;
(27) 2-(4-methoxystyryl)benzofuran;
(28) 2-(3,4-dimethoxystyryl)benzofuran;
(29) 5-chloro-2-(4-aminostyryl)benzofuran;
(30) 5-chloro-2-(4-methylaminostyryl)benzofuran;
(31) 5-chloro-2-(4-diethylaminostyryl)benzofuran;
(32) 5-chloro-2-(3-methoxy-4-methylaminostyryl)benzofuran;
(33) 5-chloro-2-(4-methoxystyryl)benzofuran;
(34) 5-chloro-2-(3,4-dimethoxystyryl)benzofuran;
(35) 5-methoxy-2-(4-diethylaminostyryl)benzofuran;
(36) 5-methoxy-2-(3-methoxy-4-methylaminostyryl)benzofuran;
(37) 5-methoxy-2-(4-methoxystyryl)benzofuran;
(38) 5-methoxy-2-(3,4-dimethoxystyryl)benzofuran;
(39) 5-methyl-2-(aminostyryl)benzofuran trifluoroacetate;
(40) 5-methyl-2-(4-methylaminostyryl)benzofuran trifluoroacetate;
(41) 5-methyl-2-(4-diethylaminostyryl)benzofuran;
(42) 5-methyl-2-(4-methoxystyryl)benzofuran;
(43) 5-methyl-2-(3,4-dimethoxystyryl)benzofuran;
(44) 5-(2-(2-fluoroethoxy)ethoxy)-2-(4-methylaminostyryl)benzofuran;
(45) 5-(2-(2-(2-fluoroethoxy)ethoxy)ethoxy)-2-(4-methylaminostyryl)benzofuran;
(46) 5-iodo-2-(4-methylaminostyryl)benzofuran;
(47) 5-iodo-2-(4-diethylaminostyryl)benzofuran;
(48) 5-iodo-2-(3-methoxy-4-methylaminostyryl)benzofuran;
(49) 5-iodo-2-(4-methoxystyryl)benzofuran;
(50) 5-iodo-2-(3,4-dimethoxystyryl)benzofuran;
(51) 5,6-dimethoxy-2-(4-dimethylaminostyryl)benzofuran;
(52) 5,6-dimethoxy-2-(4-diethylaminostyryl)benzofuran;
(53) 5,6-dimethoxy-2-(3-methoxy-4-methylaminostyryl)benzofuran;
(54) 5,6-dimethoxy-2-(4-methoxystyryl)benzofuran;
(55) 5,6-dimethoxy-2-(3,4-dimethoxystyryl)benzofuran;
(56) 5-hydroxy-2-(4-diethylaminostyryl)benzofuran;
(57) 6-methoxy-2-(4-diethylaminostyryl)benzofuran;
(58) 6-methoxy-2-(4-methoxystyryl)benzofuran;
(59) 6-methoxy-2-(3,4-dimethoxystyryl)benzofuran;
(60) 6-methoxy-2-(3-methoxy-4-methylaminostyryl)benzofuran;
(61) 6-methyl-2-(4-aminostyryl)benzofuran trifluoroacetate;
(62) 6-methyl-2-(4-methylaminostyryl)benzofuran trifluoroacetate;
(63) 6-methyl-2-(4-diethylaminostyrypbenzofuran;
(64) 6-methyl-2-(4-methoxystyryl)benzofuran;
(65) 6-methyl-2-(3,4-dimethoxystyryl)benzofuran;
(66) 6-(2-(2-(2-fluoroethoxy)ethoxy)ethoxy)-2-(4-methylaminostyryl)benzofuran;
(67) 6-hydroxy-2-(4-aminostyryl)benzofuran;
(68) 6-hydroxy-2-(4-methylaminostyryl)benzofuran;
(69) 6-hydroxy-2-(4-diethylaminostyryl)benzofuran;
(70) 5,6-dimethoxy-2-(4-methylaminostyryl)benzofuran;
(71) 5-(2,2)-dimethyltetrahydropyran-4-ylmethoxy)-2-(4-aminostryl)benzofuran;
(72) 5-(2,2-dimethyltetrahydropyran-4-ylmethoxy)-2-(4-methylaminostryl)benzofuran;
(73) 5-(2,2-dimethyltetrahydropyran-4-ylmethoxy)-2-(4-dimethylaminostryl)benzofuran;
(74) 6-(2,2-dimethyltetrahydropyran-4-ylmethoxy)-2-(4-aminostryl)benzofuran;
(75) 6-(2,2-dimethyltetrahydropyran-4-ylmethoxy)-2-(4-methylaminostryl)benzofuran; and
(76) 6-(2,2-dimethyltetrahydropyran-4-ylmethoxy)-2-(4-dimethylaminostryl)benzofuran.

The compound of formula (I) of the present invention can also be used in the form of a pharmaceutically acceptable salt formed with an inorganic or organic acid such as hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, and toluenesulfonic acid.

In accordance with another aspect of the present invention, there is provided a preparation method of the compound of formula (I), which comprises conducting a Honer-Emmons reaction, i.e., reacting a 2-(diethoxyphosphorylmethyl)benzofuran of formula (II) with an aldehyde of formula (III) in an organic solvent in the presence of a base:

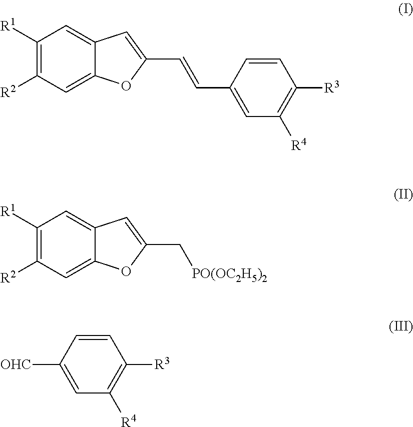

wherein, R¹, R², R³ and R⁴ have the same meanings as defined above.

The preparation method of the compound of formula (I) is depicted in Reaction Scheme 1:

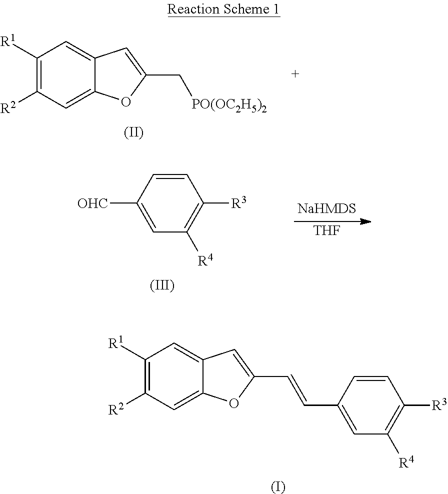

wherein, R¹, R², R³ and R⁴ have the same meanings as defined above.

Specifically, the compound of formula (I) may be prepared by allowing the 2-(diethoxyphosphorylmethyl)benzofuran of formula (II) to react with the substituted aldehyde of formula (III) in an organic solvent in the presence of a base at a temperature ranging from 0° C. to room temperature, as shown in Reaction Scheme 1.

The base which may be used in this reaction is an alkali metal hydride (e.g., NaH, LiH, KH), an alkyl alkali metal compound (e.g., n-BuLi), an alkali metal alkoxide (e.g., sodium methoxide, sodium ethoxide, sodium isopropoxide, sodium t-butoxide, potassium t-butoxide, potassium isopropoxide, lithium isopropoxide), an alkali metal amides (e.g., lithium diisopropylamide (LiN(i-Pr)₂), lithium hexamethyldisilylamide (LiHMDS), potassium hexamethyldisilylamide (KHMDS), sodium hexamethyldisilylamide (NaHMDS)) or a mixture thereof, among which potassium t-butoxide and sodium hexamethyldisilylamide are preferred.

The organic solvent suitable for use therein is ether such as tetrahydrofuran, diethyl ether, and diisopropyl ether.

2-(Diethoxyphosphorylmethyl)benzofuran of formula (II) used as a starting material in the above reaction may be prepared in accordance with anyone of the conventional procedures such as the method described in Asharm, M. J. Chem. Soc. Perkin Trans., 2, 1662 (2002); Michaelis, A. et. al., Chem. Ber., 31, 1048(1898), and Bhattacharya, A. K. et. al., Chem. Rew., 81, 415 (1981), which is shown in Reaction Scheme 2:

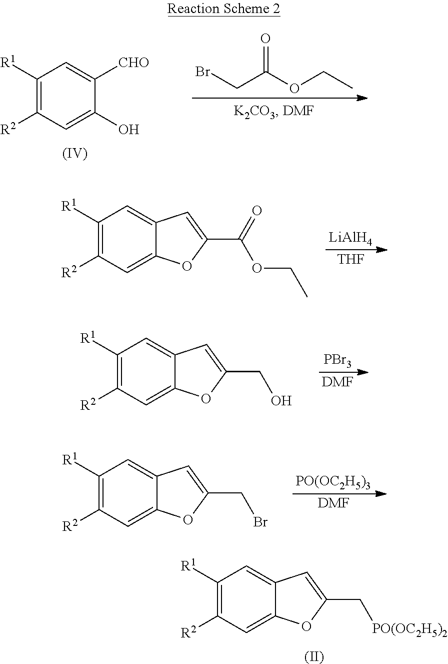

wherein, R¹ and R² have the same meanings as defined above.

Specifically, in Reaction Scheme 2,2-(diethoxyphosphorylmethyl)benzofuran is prepared by conducting a sequence of reactions: an intramolecular Aldo/Perkin type condensation of 2-hydroxybenzaldehyde of formula (IV) with ethyl bromoacetate in the presence of a base; a reduction reaction using lithium aluminium hydride; a bromination reaction using phosphorous tribromide; and a reaction with triethylphosphite.

Examples of the preferred aldehyde compound of formula (IV) include compounds of formulae (4a) to (4o):

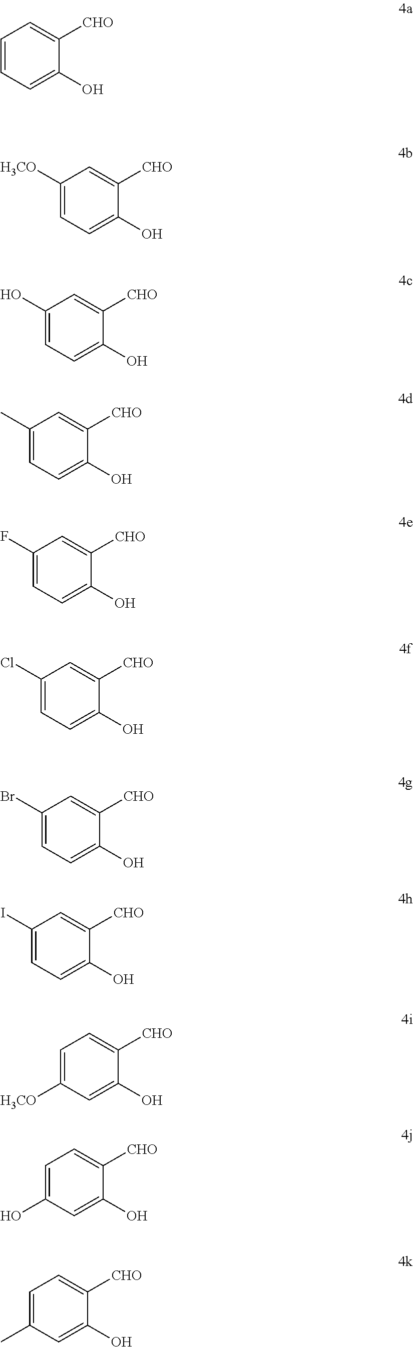

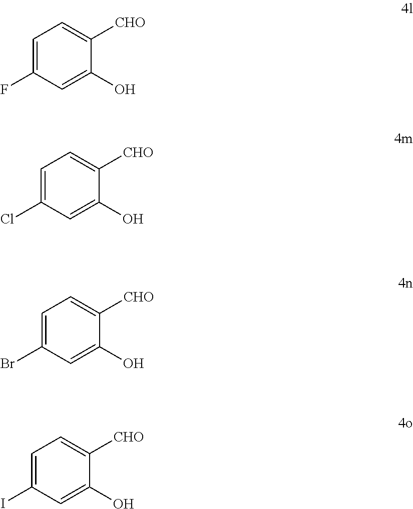

In addition, the compound prepared according to Reaction Scheme 1 is subsequently subjected to de-methylation using boron trichloride, boron trifluoride, boron tribromide, or iodotrimethylsilane, preferably boron tribromide dissolved in an organic solvent such as dichloromethane at a temperature ranging from −78° C. to room temperature for 3 to 5 hrs, to obtain the inventive compound (3), (10), (18), or (19), as shown in Reaction Scheme 3:

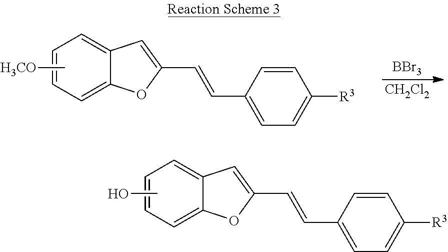

wherein, R³ has the same meaning as defined above.

The inventive compound of formula (I) or a pharmaceutically acceptable salt thereof efficiently inhibits the formation of beta-amyloid fibrils and exhibits a high degree of brain blood barrier penetrating ability, thereby effectively inhibiting the beta-amyloid fibril accumulation in the brain. Thus, the inventive compound or a pharmaceutically acceptable salt thereof are useful for preventing or treating a degenerative brain disease.

Therefore, the present invention provides a phamarceutical composition comprising the compound of formula (I), or the pharmaceutically acceptable salt thereof as an active ingredient for inhibiting the formation of beta-amyloid fibrils.

The present invention also provides a pharmaceutical composition comprising the compound of formula (I) or a pharmaceutically acceptable salt thereof as an active ingredient for preventing or treating a degenerative brain disease.

As used herein, the term “a degenerative brain disease” refers to a disease caused by the accumulation of beta-amyloid fibrils in the brain and exemplary disease include senile dementia (e.g., dementia of Alzheimers type), cerebral apoplexy, Parkinson's disease, and Huntington's disease

The pharmaceutical composition comprises the compound of formula (I) or a pharmaceutically acceptable salt thereof in an amount of 0.5 to 10% by weight, preferably 0.5 to 5% by weight, based on the total weight of the pharmaceutical composition.

The inventive pharmaceutical composition may be optionally sterilized and may further comprise a juvantia such as preservatives, stabilizer, wettable powder, emulsify promoter, salt for osmotic regulation, buffer, and other therapeutically active compounds. The inventive pharmaceutical composition may be formulated in accordance with the conventional methods such as mixing, granulation or coating in the form for oral administration or for parenteral administration

Exemplary formulations for oral administration include tablet, pilula, hard or soft-capsule, solution, emulsion, emusifier, syrup, and granule. These formulations may comprise diluent (e.g., lactose, dextrose, sucrose, mannitol, sorbitol, cellulose, glycin), lubricant (e.g., silica, talc, stearic acid and its magnesium or calsium salt, and polyethyleneglycol) as well as the above active ingredients.

The tablet may comprise a binder (e.g., magnesium aluminium silicate, starch paste, gelatin, tragacanth, methylcellulose, sodium carboxymethylcellulose, polyvinylpyrrolidine) and optionally an disintegrant or its effervescent mixture (e.g., starch, agar, and alginic acid or its sodium salt), absorber, colorant, cordial, and sweetening agent

Also, exemplary formulations for parenteral administration include an isotonic solution or a suspension for injective administration.

The inventive compound or a pharmaceutically acceptable salt thereof may be administered orally or parenterally as an active ingredient in an effective amount ranging from about 0.1 to 30 mg/kg, preferably 0.5 to 10 mg/kg body weight per day in case of mammals including human in a single dose or in divided doses.

The following Preparation Examples and Examples are intended to further illustrate the present invention without limiting its scope.

### Preparation Example 1

### 5-Methoxy-2-(diethoxyphosphorylmethyl)benzofuran (Compound of Formula 2)

### Step 1: Ethyl 5-methoxy-2-benzofuran carboxylate

To 7.61 g (0.05 mol) of 5-methoxy-2-hydroxybenzaldehyde (Compound of formula 4b) dissolved in dimethylformamide, a molecular sieve and 15.2 g (0.11 mol) of potassium carbonate were added to obtain a mixture. After adding thereto 16.7 g (0.10 mol) of ethyl bromoacetate, the mixture was refluxed at 140° C. for 40 min. Then, 15.2 g (0.11 mol) of potassium carbonate was added thereto and the resulting mixture was refluxed for 50 min. After the completion of the reaction, the molecular sieve and the resulting precipitate were isolated by filtering and the solid was washed with ethyl acetate. The wash solution and the filtrate were combined. The resulting solution was distilled under a reduced pressure, extracted with a mixture of water and ethyl acetate. The organic layer was separated, dried over anhydrous sodium sulfate, filtered, and distilled under a reduced pressure. The residue thus obtained was purified by column chromatography (n-hexane/ethyl acetate=9/1) to obtain 8.48 g of the title compound (yield: 77%).

¹H NMR (CDCl₃, 400 MHz) δ 7.42 (m, 2H), 7.02 (m, 2H), 4.39 (q, 2H, J=7.1 Hz), 3.79 (s, 3H), 1.38 (t, 3H, J=7.1 Hz).

### Step 2: 5-Methoxy-2-hydroxymethylbenzofuran

Lithium aluminium hydride (0.85 g, 22.5 mmol) was dissolved in dimethylformamide at 0° C. and 6.61 g (0.03 mol) of ethyl 5-methoxy-2-benzofuran carboxylate obtained in Step 1 in the form of a tetrahydrofuran solution was added thereto, followed by stirring at 0° C. for 10 min. After the completion of the reaction, saturated sodium sulfate was added to the resulting mixture at 0° C., and the resulting precipitate was removed by filtering. The filtrate was distilled under a reduced pressure to remove the solvent, and the residue was extracted with water and ethyl acetate. The organic layer was separated, dried over anhydrous sodium sulfate, distilled under a reduced pressure. The residue thus obtained was purified by column chromatography (n-hexane/ethyl acetate=3/1) to obtain 4.81 g of the title compound (yield: 90%).

¹H NMR (CDCl₃, 400 MHz) δ 7.31 (d, 1H, J=8.9 Hz), 6.96 (s, 1H), 6.86 (dd, 1H, J=1.7, 8.9 Hz), 6.53 (s, 1H), 4.69 (s, 2H), 3.81 (s, 3H), 2.89 (s, 1H).

### Step 3: 5-Methoxy-2-(diethoxyphosphorylmethyl)benzofuran

Phosphorous tribromide (8.12 g, 0.03 mol) was added to dimethylformamide at 0° C., followed by stirring at 0° C. for 30 min. 3.56 g (0.02 mol) of 5-methoxy-2-hydroxymethylbenzofuran obtained in Step 2 in the form of a dimethylformamide solution was added thereto, followed by stirring at 0° C. for 1 hr. After completion of the reaction, sodium carbonate and ethyl acetate were added to the reaction mixture to neutralize to pH 7-8. The resulting precipitate was isolated by filtering and the solid was washed with ethyl acetate. The wash solution and the filtrate were combined. The resulting solution was extracted with a mixture of water and ethyl acetate. The organic layer was separated, dried over anhydrous sodium sulfate, filtered, and distilled under a reduced pressure to obtain 5-methoxy-2-bromomethylbenzofuran compound. Triethylphospite was added thereto, followed by heating with reflux for 3 hrs. After the completion of the reaction, toluene was added to the reaction mixture and distilled under a reduced pressure. The residue thus obtained was purified by column chromatography (n-hexane/ethyl acetate=1/1→ethyl acetate) to obtain 5.07 g of the title compound (yield: 85%).

¹H NMR (CDCl₃, 400 MHz) δ 7.31 (d, 1H, J=8.9 Hz), 6.97 (d, 1H, J=2.6 Hz), 6.84 (dd, 1H, J=2.6, 8.9 Hz), 6.58 (d, 1H, Hz) 4.10 (qn, 4H, J=7.1 Hz), 3.82 (s, 3H), 3.35 (d, 2H, J=21.3 Hz), 1.30 (t, 6H, J=7.1 Hz).

### Example 1

### 5-Methoxy-2-(4-dimethylaminostyryl)benzofuran (Compound 2)

1.05 Equivalent amount of 1M sodium hexamethyldisilylamide (NaHMDS) tetrahydrofuran solution was added to 0.30 g (0.001 mol) of 5-methoxy-2-(diethoxyphosphorylmethyl)benzofuran obtained in Preparation Example 1 in the form of a tetrahydrofuran solution at 0° C., followed by stirring for 30 min. 0.16 g (1.05 mmol) of 4-dimethylbenzaldehyde in the form of a tetrahydrofuran solution was added thereto, followed by stirring at room temperature for 2 hrs. After completion of the reaction, methanol was added to the resulting mixture at 0° C. The resulting mixture was distilled under a reduced pressure and the residue was extracted with a mixture of water and ethyl acetate. The organic layer was separated, dried over anhydrous sodium sulfate, filtered and distilled under a reduced pressure to remove the solvent. The residue thus obtained was recrystallized to obtain 0.23 g of the title compound (yield: 80%).

¹H NMR (CDCl₃, 400 MHz) δ 7.43 (d, 2H, J=8.8 Hz), 7.33 (d, 1H, J=9.0 Hz), 7.23 (d, 1H, J=16.1 Hz), 6.97 (d, 1H, J=2.5 Hz), 6.84 (dd, 1H, J=2.6, 8.8 Hz), 6.79 (d, 1H, J=16.1 Hz), 6.72 (d, 2H, J=8.8 Hz), 6.52 (s, 1H), 3.85 (s, 3H), 3.01 (s, 6H).

¹³C NMR (CDCl₃, 100 MHz) δ 157.0, 155.9, 150.4, 149.7, 130.5, 130.0, 127.9, 124.8, 112.3, 112.1, 111.0, 103.4, 103.1, 55.9, 40.4.

### Example 2

### 5-Hydroxy-2-(4-dimethylaminostyryl)benzofuran (Compound 3)

10.0 equivalent amount of 1 M boron tribromide dichloromethane solution was added to 146.7 mg (0.5 mmol) of 5-Methoxy-2-(4-dimethylaminostyryl)benzofuran (Compound of formula 2) obtained in Example 1 in the form of a dichloromethane solution at −78° C., followed by stirring at room temperature for 3 hrs. After completion of the reaction, sodium carbonate was added thereto at 0° C. to neutralize to pH 7-8. The resulting solution was extracted with a mixture of water and dichloromethane. The organic layer was separated, dried over anhydrous sodium sulfate, filtered, and distilled under a reduced pressure to remove the solvent. Ethyl acetate was added to the residue thus obtained, followed by adding 2N HCl thereto at 0° C. The resulting precipitate was isolated by filtering, washed with ethyl acetate and dissolved in water. The resulting solution was neutralized with potassium bicarbonate at 0° C., extracted with ethyl acetate, dried over anhydrous sodium sulfate, and distilled under a reduced pressure to remove solvent. The residue thus obtained was recrystallized with methanol to obtain 69.8 g of the title compound (yield: 50%).

Mp: 177.0-178.0° C.

IR (KBr): 3436, 1602, 1520, 1358, 1197, 810 cm⁻¹

¹H NMR (DMSO-d₆, 400 MHz) δ 9.10 (s, 1H), 7.43 (d, 2H, J=7.8 Hz), 7.27 (d, 1H, J=8.5 Hz), 7.09 (d, 1H, J=16.1 Hz), 6.90 (d, 1H, J=16.1 Hz), 6.84 (s, 1H), 6.67 (m, 4H), 2.92 (s, 3H).

¹³C NMR (DMSO-d₆, 100 MHz) δ 156.6, 153.8, 150.8, 148.7, 130.5, 130.3, 128.4, 127.4, 113.1, 112.6, 112.3, 111.1, 105.5, 103.9, 40.0.

MS m/z 279 (M⁴).

### Examples 3 to 70

The procedures of Examples 1 and/or 2 were repeated by employing respective corresponding starting compounds to obtain the respective title compounds of Examples 3 to 70 having the following analytical data.

### Example 3

### 2-(4-dimethylaminostyryl)benzofuran (Compound 1)

¹H NMR (CDCl₃, 400 MHz) δ 7.49-7.43 (m, 4H), 7.28-7.16 (m, 3H), 6.82 (d, 1H, J=16.1 Hz), 6.73 (d, 2H, J=8.8 Hz), 6.57 (s, 1H), 3.01 (s, 6H).

¹³C NMR (CDCl₃, 100 MHz) δ 156.1, 154.7, 150.7, 130.7, 129.5, 127.9, 124.8, 123.9, 122.7, 120.4, 112.3, 112.1, 110.7, 103.2, 40.4.

### Example 4

### 5-methyl-2-(4-dimethylaminostyryl)benzofuran (Compound 4)

Mp: 188.0-189.0° C.

IR (KBr): 3437, 1600, 1518, 1359, 1184, 814 cm⁻¹

¹H NMR (CDCl₃, 400 MHz) δ 7.43 (d, 2H, J=8.8 Hz), 7.32 (d, 1H, J=8.3 Hz), 7.28 (s, 1H), 7.23 (d, 1H, J=16.1 Hz), 7.04 (d, 1H, J=8.3 Hz), 6.79 (d, 1H, J=16.1 Hz), 6.72 (d, 2H, J=8.8 Hz), 6.50 (s, 1H), 3.00 (s, 6H), 2.42 (s, 3H).

¹³C NMR (CDCl₃, 100 MHz) δ 156.2, 153.1, 150.4, 132.4, 130.4, 129.6, 127.9, 125.1, 124.9, 120.3, 112.3, 112.2, 110.1, 103.0, 40.4, 21.3.

MS m/z 277 (M⁺).

### Example 5

### 5-fluoro-2-(4-dimethylaminostyryl)benzofuran (Compound 5)

¹H NMR (CDCl₃, 400 MHz) δ 7.43 (d, 2H, J=8.8 Hz), 7.35 (dd, 1H, J=4.1, 8.9 Hz), 7.26 (d, 1H, J=16.1 Hz), 7.14 (dd, 1H, J=2.6, 8.6 Hz), 6.94 (td, 1H, J=2.6, 9.0 Hz), 6.78 (d, 1H, J=6.78 Hz), 6.72 (d, 2H, J=8.8 Hz), 6.52 (s, 1H), 3.01 (s, 6H).

¹³C NMR (CDCl₃, 100 MHz) δ 160.4, 158.1, 157.9, 150.9, 150.6, 131.4, 130.4, 130.3, 128.1, 124.5, 112.3, 111.7, 111.3, 111.1, 111.0, 106.0, 105.7, 103.2, 103.1, 40.4.

### Example 6

### 5-chloro-2-(4-dimethylaminostyryl)benzofuran (Compound 6)

¹H NMR (CDCl₃, 400 MHz) δ 7.45 (d, 1H, J=2.1 Hz), 7.43 (d, 2H, J=8.8 Hz), 7.35 (d, 1H, J=8.7 Hz), 7.26 (d, 1H, J=16.2 Hz), 7.16 (dd, 1H, J=2.1, 8.6 Hz), 6.77 (d, 1H, J=16.1 Hz), 6.72 (d, 2H, J=8.8 Hz), 6.50 (s, 1H), 3.01 (s, 6H).

¹³C NMR (CDCl₃, 100 MHz) δ 157.6, 153.1, 150.6, 131.7, 130.9, 128.2, 128.1, 124.5, 123.9, 117.9, 112.3, 111.9, 111.5, 102.4, 40.3.

### Example 7

### 5-bromo-2-(4-dimethylaminostyryl)benzofuran (Compound 7) ¹H NMR (CDCl₃, 400 MHz) δ 7.60 (s, 1H), 7.42 (d, 2H, J=8.4 Hz), 7.30-7.24 (m, 3H), 6.77 (d, 1H, J=16.1 Hz), 6.71 (d, 2H, J=8.3 Hz), 6.49 (s, 1H), 3.01 (s, 6H).

¹³C NMR (CDCl₃, 100 MHz) δ 157.5, 153.4, 150.8, 131.8, 131.6, 128.1, 126.6, 124.4, 122.9, 115.7, 112.3, 112.0, 111.4, 102.3, 40.3.

### Example 8

### 5-iodo-2-(4-dimethylaminostyryl)benzofuran (Compound 8)

¹H NMR (CDCl₃, 400 MHz) δ 7.81 (d, 1H, J=1.6 Hz), 7.49 (dd, 1H, J=1.7, 8.5 Hz), 7.43 (d, 2H, J=8.8 Hz), 7.26 (d, 1H, J=16.1 Hz), 7.21 (d, 1H, J=8.6 Hz), 6.77 (d, 1H, J=16.2 Hz), 6.72 (d, 2H, J=8.8 Hz), 6.48 (s, 1H), 3.01 (s, 6H).

¹³C NMR (CDCl₃, 100 MHz) δ 157.1, 154.1, 150.6, 132.3, 131.8, 129.1, 128.1, 127.9, 124.4, 112.6, 112.2, 111.4, 101.9, 86.2, 40.3.

### Example 9

### 6-methoxy-2-(4-dimethylaminostyryl)benzofuran (Compound 9)

Mp: 194.5-195.5° C.

IR (KBr): 3437, 1602, 1489, 1356, 1146, 1107, 820 cm⁻¹

¹H NMR (CDCl₃, 400 MHz) δ 7.43 (d, 2H, J=8.8 Hz), 7.33 (d, 1H, J=9.0 Hz), 7.23 (d, 1H, J=16.1 Hz), 6.97 (d, 1H, J=2.5 Hz), 6.84 (dd, 1H, J=2.6, 8.8 Hz), 6.79 (d, 1H, J=16.1 Hz), 6.72 (d, 2H, J=8.8 Hz), 6.52 (s, 1H), 3.85 (s, 3H), 3.01 (s, 6H).

¹³C NMR (CDCl₃, 100 MHz) δ 157.0, 155.9, 150.4, 149.7, 130.5, 130.0, 127.9, 124.8, 112.3, 112.1, 111.0, 103.4, 103.1, 55.9, 40.4.

MS m/z 293 (M⁺).

### Example 10

### 6-hydroxy-2-(4-dimethylaminostyryl)benzofuran (Compound 10)

¹H NMR (DMSO-d₆, 400 MHz) δ 9.53 (s, 1H), 7.41 (d, 2H, J=8.3 Hz), 7.30 (d, 1H, J=8.3 Hz), 7.01 (d, 1H, J=16.1 Hz), 6.88 (m, 2H), 6.67 (m, 4H), 2.91 (s, 3H).

¹³C NMR (DMSO-d₆, 100 MHz) δ 156.1, 155.8, 154.7, 150.6, 128.9, 128.1, 124.7, 121.5, 121.1, 112.7, 112.5, 112.1, 104.0, 97.8, 40.0.

### Example 11

### 6-methyl-2-(4-dimethylaminostyryl)benzofuran (Compound 11)

¹H NMR (CDCl₃, 400 MHz) δ 7.44 (d, 2H, J=8.5 Hz), 7.38 (d, 1H, J=7.8 Hz), 7.24 (m, 2H), 7.03 (d, 1H, J=7.8 Hz), 6.81 (d, 1H, J=16.1 Hz), 6.75 (d, 2H, J=7.7 Hz), 6.53 (s, 1H), 3.01 (s, 6H), 2.48 (s, 3H).

¹³C NMR (CDCl₃, 100 MHz) δ 155.6, 155.2, 150.2, 134.3, 129.9, 127.9, 127.0, 125.2, 124.1, 119.9, 112.5, 111.0, 103.3, 40.5, 21.8.

### Example 12

### 6-fluoro-2-(4-dimethylaminostyryl)benzofuran (Compound 12)

¹H NMR (CDCl₃, 400 MHz) δ 7.44-7.36 (m, 3H), 7.22 (d, 1H, J=16.2 Hz), 7.17 (dd, 1H, J=1.6, 9.0 Hz), 6.95 (m, 1H), 6.77 (d, 1H, J=16.2 Hz), 6.72 (d, 2H, J=8.9 Hz), 6.53 (s, 1H), 3.01 (s, 6H).

¹³C NMR (CDCl₃, 100 MHz) δ 161.9, 159.5, 157.0 (d, 2C), 154.7, 154.6, 150.5, 130.6, 127.9, 125.7; 124.7, 120.5, 120.4, 112.3, 111.8, 110.9, 110.7, 102.7, 98.8, 98.5, 40.4.

### Example 13

### 6-chloro-2-(4-dimethylaminostyryl)benzofuran (Compound 13)

¹HNMR (CDCl₃, 400 MHz) δ 7.43 (m, 3H), 7.39 (d, 1H, J=8.3 Hz), 7.24 (d, 1H, J=16.1 Hz), 7.16 (dd, 1H, J=1.8, 8.3 Hz), 6.77 (d, 1H, J=16.1 Hz), 6.72 (d, 2H, J=8.9 Hz), 6.52 (s, 1H), 3.01 (s, 6H).

¹³C NMR (CDCl₃, 100 MHz) δ 157.0, 154.7, 150.5, 131.3, 129.4, 128.2, 128.0, 124.5, 123.4, 120.8, 112.3, 111.5, 111.2, 102.7, 40.3.

### Example 14

### 6-bromo-2-(4-dimethylaminostyryl)benzofuran (Compound 14)

¹H NMR (CDCl₃, 400 MHz) δ 7.59 (s, 1H), 7.42 (d, 2H, J=8.7 Hz), 7.34-7.22 (m, 3H), 6.76 (d, 1H, J=16.1 Hz), 6.70 (d, 2H, J=8.6 Hz), 6.50 (s, 1H), 3.00 (s, 6H).

¹³C NMR (CDCl₃, 100 MHz) δ 156.9, 155.0, 150.6, 131.4, 128.6, 128.1, 126.1, 124.5, 121.9, 116.9, 114.1, 112.3, 111.5, 102.7, 40.3.

### Example 15

### 6-iodo-2-(4-dimethylaminostyryl)benzofuran (Compound 15)

¹H NMR (CDCl₃, 400 MHz) δ 7.80 (s, 1H), 7.48 (dd, 1H, J=1.3, 8.1 Hz), 7.43 (d, 2H, J=8.8 Hz), 7.24 (m, 2H), 6.77 (d, 1H, J=16.1 Hz), 6.72 (d, 2H, J=8.8 Hz), 6.51 (s, 1H), 3.01 (s, 6H).

¹³C NMR (CDCl₃, 100 MHz) δ 156.6, 155.2, 150.6, 131.7, 131.5, 129.2, 128.1, 124.5, 121.7, 119.9, 112.3, 111.4, 102.7, 86.9, 40.3.

### Example 16

### 5-methoxy-2-(4-aminostyryl)benzofuran (Compound 16)

¹H NMR (CDCl₃, 400 MHz) δ 7.38 (d, 1H, J=8.9 Hz), 7.29 (d, 2H, J=8.5 Hz), 7.07 (m, 2H), 6.86 (d, 1H, J=16.2 Hz), 6.80 (dd, 1H, J=2.6, 8.9 Hz), 6.68 (s, 1H), 6.55 (d, 2H, J=8.5 Hz), 5.44 (s, 2H), 3.75 (s, 3H).

¹³C NMR (CDCl₃, 100 MHz) δ 156.6, 155.9, 149.8, 146.7, 130.3, 129.9, 128.1, 127.2, 115.2, 113.0, 112.6, 111.1, 103.8, 103.1, 55.9.

### Example 17

### 5-methoxy-2-(4-methylaminostyryl)benzofuran (Compound 17)

Mp: 174.0-175.0° C.

IR (KBr): 3409, 1602, 1519, 1201, 1183, 819 cm⁻¹

¹H NMR (CDCl₃, 400 MHz) δ 7.38 (d, 2H, J=8.6 Hz), 7.33 (d, 1H, J=8.9 Hz), 7.2 (d, 1H, J=16.1 Hz), 6.97 (d, 1H, J=2.5 Hz), 6.83 (dd, 1H, J=2.6, 8.8 Hz), 6.77 (d, 1H, J=16.1 Hz), 6.61 (d, 2H, J=8.5 Hz), 6.51 (s, 1H), 3.85 (s, 3H), 2.88 (s, 3H), 1.55 (s, 3H).

¹³C NMR (CDCl₃, 100 MHz) δ 156.8, 155.8, 149.7, 149.3, 130.5, 130.0, 128.0, 125.8, 112.4, 112.4, 111.0, 103.4, 103.0, 55.9, 30.5.

MS m/z 279 (M⁺).

### Example 18

### 5-hydroxy-2-(4-aminostyryl)benzofuran hydrochloride (Compound 18)

¹H NMR (MeOD-d₄, 400 MHz) δ 7.74 (d, 2H, J=8.5 Hz), 7.39 (d, 2H, J=8.5 Hz), 7.28 (m, 2H), 7.19 (d, 1H, J=16.2 Hz), 6.91 (d, 1H, J=2.4 Hz), 6.78 (dd, 1H, J=2.5, 8.8 Hz), 6.73 (s, 1H).

¹³C NMR (MeOD-d₄, 100 MHz) δ 155.1, 153.2, 149.6, 137.8, 129.9, 129.7, 127.8, 127.3, 123.0, 118.3, 113.5, 110.5, 106.0, 105.1.

### Example 19

### 5-hydroxy-2-(4-methylaminostyryl)benzofuran hydrochloride (Compound 19)

¹H NMR (MeOD-d₄, 400 MHz) δ 7.77 (d, 2H, J=8.6 Hz), 7.48 (d, 2H, J=8.6 Hz), 7.29 (m, 2H), 7.21 (d, 1H, J=16.2 Hz), 6.91 (d, 1H, J=2.4 Hz), 6.79 (dd, 1H, J=2.5, 8.8 Hz), 6.74 (s, 1H), 3.09 (s, 1H).

¹³C NMR (MeOD-d₄, 100 MHz) δ 155.1, 153.2, 149.6, 138.3, 136.3, 129.7, 128.0, 127.1, 121.9, 118.5, 113.6, 110.5, 106.2, 105.1, 36.2.

### Example 20

### 6-methoxy-2-(4-aminostyryl)benzofuran (Compound 20)

¹H NMR (CDCl₃, 400 MHz) δ 7.35 (m, 3H), 7.15 (d, 1H, J=16.1 Hz), 7.02 (d, 1H, J=1.7 Hz), 6.83 (dd, 1H, J=2.2, 8.5 Hz), 6.78 (d, 1H, J=16.1 Hz), 6.68 (d, 2H, J=8.4 Hz), 6.51 (s, 1H), 3.86 (s, 3H), 3.79 (br s, 2H).

¹³C NMR (CDCl₃, 100 MHz) δ 158.0, 155.8, 155.1, 146.5, 129.1, 127.9, 127.4, 122.8, 120.6, 115.2, 113.0, 111.5, 103.6, 95.7, 55.7.

### Example 21

### 6-methoxy-2-(4-methylaminostyryl)benzofuran (Compound 21)

¹H NMR (CDCl₃, 400 MHz) δ 7.37 (m, 3H), 7.17 (d, 1H, J=16.1 Hz), 7.02 (d, 1H, J=2.0 Hz), 6.83 (dd, 1H, J=2.2, 8.5 Hz), 6.77 (d, 1H, J=16.1 Hz), 6.61 (d, 2H, J=8.6 Hz), 6.46 (s, 1H), 3.87 (s, 3H), 2.88 (s, 3H).

¹³C NMR (CDCl₃, 100 MHz) δ 157.9, 155.8, 155.4, 149.3, 129.4, 127.9, 126.1, 122.8, 120.5, 112.4, 112.3, 111.4, 103.2, 95.7, 55.7, 30.6.

### Example 22

### 5-methoxy-2-(3-methoxy-4-dimethylaminostyryl)benzofuran (Compound 22)

Mp: 226.5-227.5° C.

IR (KBr): 3436, 1595, 1507, 1470, 1204, 1167, 834 cm⁻¹

¹H NMR (CDCl₃, 400 MHz) δ 7.36-7.33 (m, 1H), 7.10-7.07 (m, 1H), 7.03 (d, J=1.69 Hz, 1H), 6.99 (d, J=2.55 Hz, 1H) 6.93-6.91 (m, 1H), 6.58 (s, 1H), 3.96 (s, 3M), 3.85 (s, 3H), 2.83 (s, 6H).

¹³C NMR (CDCl₃, 100 MHz) δ 156.29, 155.98, 152.29, 149.83, 142.84, 130.76, 130.23, 129.83, 129.65, 120.21, 118.01, 117.44, 114.47, 113.81, 104.46, 103.27, 103.17, 55.90, 55.39, 43.20.

MS m/z 323 (M⁺).

### Example 23

### 6-methoxy-2-(3-methoxy-4-dimethylaminostyryl)benzofuran (Compound 23)

¹H NMR (CDCl₃, 400 MHz) δ 7.38 (d, J=8.46 Hz, 1H), 7.19 (d, J=16.09 Hz), 7.02 (s, 2H), 6.87-6.83 (m, 2H), 6.56 (s, 1H), 6.56 (s, 1H), 3.95 (s, 3H), 3.87 (s, 3H), 2.83 (s, 6H).

¹³C NMR (CDCl₃, 100 MHz) 158.18, 155.88, 154.80, 152.31, 142.62, 131.03, 128.98, 122.65, 120.74, 120.01, 118.03, 114.55, 111.62, 108.54, 104.25, 95.74, 55.74, 55.38, 43.23.

### Example 24

### 2-(4-aminostyryl)benzofuran trifluoroacetate (Compound 24)

¹H NMR (400 MHz, MeOD) δ 7.70 (d, 2H, J=8.49 Hz), 7.57 (d, 1H, J=7.76 Hz), 7.48 (d, 1H, J=8.16 Hz), 7.35-7.29 (m, 4H), 7.22 (t, 1H, J=7.49 Hz), 7.20 (d, 1H, J=16.23 Hz), 6.84 (s, 1H)

### Example 25

### 2-(4-methylaminostyryl)benzofuran trifluoroacetate (Compound 25)

¹H NMR (400 MHz, MeOD) δ 7.60 (d, 2H, J=8.5 Hz), 7.52 (d, 1H, J=7.6 Hz), 7.44 (d, 1H, J=8.2 Hz), 7.26 (d, 1H, J=16.13 Hz), 7.26 (t, 1H, J=8.02 Hz), 7.18 (t, 1H, J=7.65 Hz), 7.09 (d, 2H, J=7.79 Hz), 7.07 (d, 1H, J=16.52 Hz), 6.75 (s, 1H), 2.96 (s, 3H)

### Example 26

### 2-(4-diethylaminostyryl)benzofuran (Compound 26)

¹H NMR (400 MHz, CDCl₃) δ 7.49 (d, 2H, J=7.18 Hz), 7.46-7.40 (m, 3H), 7.27-7.16 (m, 2H), 6.78 (d, 1H, J=16.06 Hz), 6.67 (d, 1H, J=7.86 Hz), 6.56 (s, 1H), 3.40 (d, 4H, J=6.68 Hz), 1.20-1.18 (m, 6H)

### Example 27

### 2-(4-methoxystyryl)benzofuran (Compound 27)

¹H NMR (400 MHz, CDCl₃) δ 7.53-7.45 (m, 4H), 7.3 7.24 (m, 2H), 7.19 (t, 1H, J=8.44 Hz), 6.92 (d, 2H, J=8.78 Hz), 6.88 (d, 1H, J=16.15 Hz), 6.63 (s, 1H), 3.83 (s, 3H)

### Example 28

### 2-(3,4-dimethoxystyryl)benzofuran (Compound 28)

¹H NMR (400 MHz, CDCl₃) δ 7.52 (d, 1H, J=7.48 Hz), 7.46 (d, 1H, J=8.02 Hz), 7.29-7.24 (m, 3H), 7.19 (t, 1H, J=8.73 Hz), 7.10-7.08 (m, 2H), 6.88 (d, 2H, J=8.12 Hz, J=4.6 Hz), 6.64 (s, 1H), 3.94 (d, 6H, J=9.32 Hz)

### Example 29

### 5-chloro-2-(4-aminostyryl)benzofuran (Compound 29)

¹H NMR (400 MHz, DMSO) δ 7.63 (d, J=1.93 Hz, 1H), 7.54 (d, J=8.64 Hz, 1H), 7.43 (m, J=8.35 Hz, 2H), 7.25 (dd, J=8.69 Hz, J=2.03 Hz, 1H), 7.18 (d, J=16.22 Hz, 1H), 7.00 (d, J=16.27 Hz, 1H), 6.80 (s, 1H), 6.75 (d, J=8.15 Hz, 2H), 3.93 (s, 2H)

### Example 30

### 5-chloro-2-(4-methylaminostyryl)benzofuran (Compound 30)

¹H NMR (400 MHz, DMSO) δ 7.91 (d, J=1.93 Hz, 1H), 7.50 (dd, J=6.72 Hz, J=1.79 Hz, 1H), 7.36 (m, 3H), 7.15 (d, J=16.19 Hz, 1H), 7.91 (d, J=16.20 Hz, 1H), 6.72 (s, 1H), 6.53 (d, J=8.64 Hz, 2H), 6.08 (q, J=5.01 Hz, 1H), 2.70 (d, J=5.01 Hz, 3H)

### Example 31

### 5-chloro-2-(4-diethylaminostyryl)benzofuran (Compound 31)

¹HNMR (400 MHz, CDCl₃) δ 7.65 (d, J=2.12 Hz, 1H), 7.40 (d, J=8.82 Hz, 2H), 7.34 (d, J=8.6 Hz 1H), 7.18 (s, 1H), 6.77 (s, 1H), 6.72 (d, J=2.97 Hz 1H), 6.67 (d, J=8.87 Hz 2H), 6.48 (s, 1H), 3.40 (q, J=7.08 Hz, 4H), 1.19 (t, J=7.02 Hz, 6H)

### Example 32

### 5-chloro-2-(3-methoxy-4-methylaminostyryl)benzofuran (Compound 32)

¹H NMR (400 MHz, DMSO) δ 7.63 (dd, J=13.45 Hz, J=1.79 Hz, 1H), 7.52 (t, J=9 Hz, 1H); 7.13 (m, 3H), 6.82 (m, 1H), 6.58 (d, J=13.09 Hz, 1H), 6.45 (d, J=8.16 Hz, 1H), 6.25 (d, J=12.9 Hz, 1H), 5.46 (q, J=5.76 Hz, 1H), 3.78 (s, 3H) 2.73 (d, J=5.01 Hz, 3H)

### Example 33

### 5-chloro-2-(4-methoxystyryl)benzofuran (Compound 33)

¹H NMR (400 MHz, CDCl₃) δ 7.48 (d, J=2.34 Hz, 1H), 7.46 (d, J=4.12 Hz, 1H), 7.37 (m, J=8.64 Hz, 1H), 7.28 (d, J=15.86 Hz, 1H), 7.20 (dd, J=8.65 Hz, J=2.10 Hz, 1H), 6.92 (d, J=8.74 Hz, 2H), 6.84 (d, J=16.15 Hz, 1H), 6.56 (s, 1H), 3.85 (s, 3H)

### Example 34

### 5-chloro-2-(3,4-dimethoxystyryl)benzofuran (Compound 34)

¹H NMR (400 MHz, CDCl₃) δ 7.47 (d, J=2.01 Hz, 1H), 7.36 (d, J=8.61 Hz, 1H), 7.27 (m, J=16.11 Hz, 1H), 7.20 (dd, J=8.64 Hz, J=1.91 Hz, 1H), 7.08 (m, 2H), 6.85 (m, 2H), 6.57 (s, 1H), 3.95 (s, 3H), 3.93 (s, 3H)

### Example 35

### 5-methoxy-2-(4-diethylaminostyryl)benzofuran (Compound 35)

¹H NMR (400 MHz, CDCl₃) δ 7.40 (d, J=8.8 Hz, 2H), 7.32 (d, J=8.8 Hz, 1H), 7.16 (d, J=16 Hz, 1H), 6.96 (d, J=2.8 Hz, 1H), 6.83 (dd, J=2.4, 8.8 Hz, 1H), 6.75 (d, J=16 Hz, 1H), 6.66 (d, J=9.2 Hz, 2H), 6.50 (s, 1H), 3.84 (s, 3H), 3.39 (q, J=7.2 Hz, 4H), 1.19 (t, J=6.8 Hz, 6H).

### Example 36

### 5-methoxy-2-(3-methoxy-4-methylaminostyryl)benzofuran (Compound 36)

¹H NMR (400 MHz, CDCl₃) δ 7.35 (d, J=9.2 Hz, 1H), 7.26 (d, J=16 Hz, 1H), 7.10 (m, 2H), 7.04 (s, 1H), 6.70 (d, J=2.4 Hz, 1H), 6.94 (d, J=16 Hz, 1H), 6.88 (dd, J=2.8, 8.8 Hz, 1H), 6.63 (s, 1H), 3.89 (s, 3H), 3.85 (s, 3M), 3.15 (s, 3H).

### Example 37

### 5-methoxy-2-(4-methoxystyryl)benzofuran (Compound 37)

¹H NMR (400 MHz, CDCl₃) δ 7.46 (d, J=8.8 Hz, 2H), 7.34 (d, J=8.8 Hz, 1H), 7.24 (d, J=16.4 Hz, 1H), 6.98 (d, J=2.8 Hz, 1H), 6.91 (d, J=8.8 Hz, 2H), 6.85 (d, J=16.4 Hz, 1H), 6.84 (d, J=2.8 Hz, 1H), 6.57 (s, 1H), 3.85 (s, 3H), 3.84 (s, 3H).

### Example 38

### 5-methoxy-2-(3,4-dimethoxystyryl)benzofuran (Compound 38)

¹H NMR (400 MHz, CDCl₃) δ 7.34 (d, J=8.8 Hz, 1H), 7.24 (d, J=16 Hz, 1H), 7.09 (m, 2H), 6.99 (d, J=2.8 Hz, 1H), 6.88 (d, J=2.8 Hz, 1H), 6.87 (m, 1H), 6.85 (d, J=16 Hz, 1H), 6.58 (s, 1H), 3.95 (s, 3H), 3.91 (s, 3H), 3.85 (s, 3H).

### Example 39

### 5-methyl-2-(aminostyryl)benzofuran trifluoroacetate (Compound 39)

¹H NMR (400 MHz, MeOD) δ 7.64 (d, 2H, J=8.47 Hz), 7.32 (d, 2H, J=8.37 Hz), 7.26 (d, 1H, J=16.32 Hz), 7.23 (d, 1H, J=8.54 Hz), 7.14 (s, 1H), 7.09 (d, 1H, J=7.94, Hz), 6.72 (s, 1H), 2.40 (s, 3H)

### Example 40

### 5-methyl-2-(4-methylaminostyryl)benzofuran trifluoroacetate (Compound 40)

¹H NMR (400 MHz, MeOD) δ 7.60 (d, 2H, J=8.5 Hz), 7.31 (d, 2H, J=7.09 Hz), 7.24 (d, 1H, J=16.16 Hz), 7.13 (d, 2H, J=8.33 Hz), 7.09 (s, 1H), 7.06 (d, 1H, J=10.49 Hz), 6.68 (s, 1H), 3.00 (s, 3H), 2.38 (s, 3H)

### Example 41

### 5-methyl-2-(4-diethylaminostyryl)benzofuran (Compound 41)

¹H NMR (400 MHz, CDCl₃) δ 7.41 (d, 2H, J=8.4 Hz), 7.33 (d, 1H, J=8.27 Hz), 7.29-7.26 (m, 1H), 7.23 (d, 1H, J=16.11 Hz), 7.04 (d, 1H, J=8.08 Hz), 6.77 (d, 1H, J=16.08 Hz), 6.67 (d, 1H, J=8.38 Hz), 6.49 (s, 1H), 3.40 (d, 4H, J=6.93 Hz), 2.43 (s, 3H), 1.27-1.18 (m, 6H)

### Example 42

### 5-methyl-2-(4-methoxystyryl)benzofuran (Compound 42)

¹H NMR (300 MHz, CDCl₃) δ 7.48 (d, 2H, J=8.64 Hz), 7.34 (d, 1H, J=8.52 Hz), 7.3 7.23 (m, 2H), 7.07 (d, 1H, J=8.1 Hz), 6.92 (d, 2H, J=8.7 Hz), 6.87 (d, 1H, J=16.17 Hz), 6.57 (s, 1H), 3.86 (s, 3H), 2.43 (s, 3H)

### Example 43

### 5-methyl-2-(3,4-dimethoxystyryl)benzofuran (Compound 43)

¹H NMR (300 MHz, CDCl₃) δ 7.4 (d, 1H, J=7.85 Hz), 7.27-7.21 (m, 3H), 7.1 7.08 (m, 2H), 7.04 (d, 1H, J=7.98 Hz), 6.90 (s, 1H), 6.86 (d, 1H, J=10.808 Hz), 6.60 (s, 1H), 3.95 (d, 6H, J=7.97 Hz), 2.49 (s, 3H)

### Example 44

### 5-(2-(2-fluoroethoxy)ethoxy)-2-(4-methylaminostyryl)benzofuran (Compound 44)

¹H NMR (400 MHz, CDCl₃) δ 7.42 (d, J=8.4 Hz, 1H), 7.37 (d, J=8.4 Hz, 1H), 7.31 (d, J=8.4 Hz, 1H), 7.20 (d, J=16 Hz, 1H), 6.98 (d, J=1.6 Hz, 1H), 6.84 (dd, J=2.4, 8.8 Hz, 1H), 6.76 (d, J=16 Hz, 1H), 6.60 (d, J=8.4 Hz, 2H), 6.50 (s, 1H), 4.61 (dt, J=47.6, 4.0 Hz, 2H), 4.17 (t, J=4.8, 2H), 3.90 (m, 3H), 3.80 (t, J=4.0 Hz, 1H), 2.88 (s, 3H).

### Example 45

### 5-(2-(2-(2-fluoroethoxy)ethoxy)ethoxy)-2-(4-methylaminostyryl)benzofuran (Compound 45)

¹H NMR (400 MHz, CDCl₃) δ 7.38 (d, J=8.8 Hz, 1H), 7.34 (d, J=8.4 Hz, 1H), 7.31 (d, J=8.8 Hz, 1H), 7.20 (d, J=16 Hz, 1H), 6.98 (d, J=2.4 Hz, 1H), 6.85 (dd, J=2.8, 8.8 Hz, 1H), 6.76 (d, J=16 Hz, 1H), 6.60 (d, J=8.8 Hz, 2H), 6.50 (s, 1H), 4.57 (dt, J=47.6, 4.0 Hz, 2H), 4.16 (t, J=4.8 Hz, 2H), 3.88 (t, J=4.8 Hz, 2H), 3.76 (m, 4H), 3.62 (m, 2H), 2.10 (s, 3H).

¹³C NMR (100 MHz, CDCl₃) δ 157.09, 156.56, 155.04, 146.61, 114.66, 113.87, 113.30, 111.02, 104.27, 103.88, 83.99, 82.31, 70.84, 70.53, 70.33, 69.93, 69.20, 68.23, 30.92.

### Example 46

### 5-iodo-2-(4-methylaminostyryl)benzofuran (Compound 46)

¹H NMR (400 MHz, CDCl₃) δ 7.81 (s, 1H), 7.46 (dd, J=10.92 Hz, J=1.39 Hz, 1H), 7.37 (d, J=8.53 Hz, 2H), 7.21 (d, J=10.61 Hz, 2H), 7.07 (s, 1H), 6.76 (d, J=16.10 Hz, 1H), 6.60 (d, J=8.47 Hz, 2H), 6.49 (s, 1H), 3.95 (s, 1H), 2.95 (s, 3H)

### Example 47

### 5-iodo-2-(4-diethylaminostyryl)benzofuran (Compound 47)

¹H NMR (400 MHz, CDCl₃) δ 7.49 (d, J=6.91 Hz, 1H), 7.44 (d, J=8.04 Hz, 1H), 7.41 (d, J=8.82 Hz, 2H), 7.23 (s, 1H), 7.19 (m, 1H), 6.78 (d, J=16.19 Hz, 1H), 6.67 (d, J=8.85 Hz, 2H), 6.56 (s, 1H), 3.40 (q, J=7.07 Hz, 4H) 1.20 (t, J=7.04 Hz, 6H)

### Example 48

### 5-iodo-2-(3-methoxy-4-methylaminostyryl)benzofuran (Compound 48)

¹H NMR (400 MHz, DMSO) δ 7.52 (m, 2H), 7.21 (m, 2H), 7.01 (q, J=13.80 Hz, 1H), 6.90 (s, 1H), 6.57 (d, J=12.87 Hz, 1H), 6.45 (q, J=4.32 Hz, 1H), 6.25 (d, J=12.78 Hz, 1H), 5.41 (t, J=3.88 Hz, 1H), 3.85 (s, 1H) 3.73 (s, 2H), 2.73 (s, 3H)

### Example 49

### 5-iodo-2-(4-methoxystyryl)benzofuran (Compound 49)

¹H NMR (400 MHz, DMSO) δ 7.59 (d, J=7.55 Hz, 1H), 7.53 (d, J=7.96 Hz, 1H), 7.30 (s, 1H), 7.25 (t, J=8.24 Hz, 2H), 7.20 (d, J=4.93 Hz, 2H), 7.15 (d, J=8.2 Hz, 1H), 6.95 (d, J=8.29 Hz, 1H), 6.87 (s, 1H), 3.78 (s, 3H)

### Example 50

### 5-iodo-2-(3,4-dimethoxystyryl)benzofuran (Compound 50)

¹H NMR (400 MHz, DMSO) δ 7.59 (d, J=8.7 Hz, 2H), 7.53 (d, J=7.66 Hz, 1H), 7.26 (q, J=7.96 Hz, 1H), 7.22 (t, J=9.30 Hz, M), 7.13 (m, J=16.28 Hz, 1H), 6.96 (d, J=8.62 Hz, 1H), 6.86 (d, J=15.88 Hz, 1H), 3.82 (s, 3H), 3.77 (s, 3H)

### Example 51

### 5,6-dimethoxy-2-(4-dimethylaminostyryl)benzofuran (Compound 51)

¹H NMR (400 MHz, DMSO) δ 7.36 (d, J=18 Hz, 2H), 7.21 (d, J=9.8 Hz, 3H), 7.12 (s, 1H), 7.03 (s, 1H), 6.93 (s, 1H), 6.43 (s, 1H), 3.85 (s, 3H), 3.82 (s, 3H), 3.80 (s, 3H)

### Example 52

### 5,6-dimethoxy-2-(4-diethylaminostyryl)benzofuran (Compound 51)

¹H NMR (400 MHz, DMSO) δ 7.36 (d, J=18.12 Hz, 2H), 7.21 (d, J=9.8 Hz, 3H), 7.12 (s, 1H), 7.03 (s, 1H), 6.93 (s, 1H), 6.43 (s, 1H), 3.85 (s, 5H), 3.82 (s, 5H), 3.80 (s, 3H), 3.78 (s, 3H)

### Example 53

### 5,6-dimethoxy-2-(3-methoxy-4-methylaminostyryl)benzofuran (Compound 53)

¹H NMR (400 MHz, DMSO) δ 7.12 (s, 1H), 6.98 (d, J=6.24 Hz, 1H), 7.00 (m, 2H), 6.65 (s, 1H), 6.42 (d, J=8.2 Hz, 1H), 5.35 (q, J=4.96 Hz, 1H), 3.79 (s, 3H), 3.77 (s, 3H), 3.74 (s, 3H), 2.72 (d, J=4.88 Hz, 3H)

### Example 54

### 5,6-dimethoxy-2-(4-methoxystyryl)benzofuran (Compound 54)

¹H NMR (400 MHz, DMSO) δ 7.52 (d, J=8.62 Hz, 2H), 7.20 (s, 1H), 7.06 (s, 1H), 7.05 (s, 2H), 6.92 (d, J=8.58 Hz, 2H), 6.73 (s, 1H), 3.96 (s, 3H), 3.92 (s, 6H)

### Example 55

### 5,6-dimethoxy-2-(3,4-dimethoxystyryl)benzofuran (Compound 55)

¹H NMR (400 MHz, CDCl₃) δ 7.54 (s, 1H), 7.06 (m, 3H), 7.00 (s, 1H), 6.86 (m, 2H), 6.58 (s, 1H), 3.98 (s, 3H), 3.95 (s, 3H), 3.93 (s, 3H), 3.92 (s, 3H).

### Example 56

### 5-hydroxy-2-(4-diethylaminostyryl)benzofuran (Compound 56)

¹H NMR (400 MHz, CDCl₃) δ 7.39 (d, J=8.4 Hz, 2H), 7.28 (d, J=8.8 Hz, 1H), 7.21 (d, J=16 Hz, 1H), 6.90 (d, J=2.4 Hz, 1H), 6.74 (d, J=16 Hz, 1H), 6.72 (dd, J=2.4, 8 Hz, 1H), 6.66 (d, J=8.8 Hz, 2H), 6.46 (s, 1H), 4.59 (s, OH), 3.39 (q, J=7.2 Hz, 4H), 1.19 (t, J=6.8 Hz, 6H).

### Example 57

### 6-methoxy-2-(4-diethylaminostyryl)benzofuran (Compound 57)

¹H NMR (400 MHz, CDCl₃) δ 7.38 (d, J=8.8 Hz, 2H), 7.35 (d, J=8.4 Hz, 1H), 7.15 (d, J=16 Hz, 1H), 7.01 (d, J=2.4 Hz, 1H), 6.82 (dd, J=2.4, 8.4 Hz, 1H), 6.74 (d, J=16 Hz, 1H), 6.66 (d, J=8.8 Hz, 2H), 6.48 (s, 1H), 3.86 (s, 3H), 3.39 (q, J=6.8 Hz, 4H), 1.19 (t, J=7.2 Hz, 6H).

### Example 58

### 6-methoxy-2-(4-methoxystyryl)benzofuran (Compound 58)

¹H NMR (400 MHz, CDCl₃) δ 7.45 (d, J=8.8 Hz, 2H), 7.37 (d, J=8.4 Hz, 1H), 7.18 (d, J=16 Hz, 1H), 7.02 (d, J=2.0 Hz, 1H), 6.86 (d, J=16 Hz, 1H), 6.84 (dd, J=2.4, 8.4 Hz, 1H), 6.83 (d, J=8.8 Hz, 2H), 6.55 (s, 1H), 3.87 (s, 3H), 3.84 (s, 3H).

### Example 59

### 6-methoxy-2-(3,4-dimethoxystyryl)benzofuran (Compound 59)

¹H NMR (400 MHz, CDCl₃) δ 7.38 (d, J=8.4 Hz, 1H), 7.18 (d, J=16 Hz, 1H), 7.07 (m, 2H), 7.01 (m, 1H), 6.86 (m, 2H), 6.84 (d, J=16 Hz, 1H), 6.56 (s, 1H), 3.95 (s, 3H), 3.91 (s, 3H), 3.87 (s, 3H).

### Example 60

### 6-methoxy-2-(3-methoxy-4-methylaminostyryl)benzofuran (Compound 60)

¹H NMR (400 MHz, CDCl₃) δ 7.39 (d, J=8.8 Hz, 1H), 7.19 (d, J=16 Hz, 1H), 7.05 (m, 4H), 6.92 (d, J=16 Hz, 1H), 6.85 (dd, J=2.8, 8.8 Hz, 1H), 6.61 (s, 1H), 3.89 (s, 3H), 3.87 (s, 3H), 3.14 (s, 3H).

### Example 61

### 6-methyl-2-(4-aminostyryl)benzofuran trifluoroacetate (Compound 61)

¹H NMR (400 MHz, MeOD) δ 7.65 (d, 2H, J=8.51 Hz), 7.40 (d, 1H, J=7.92 Hz), 7.27 (s, 2H), 7.23 (d, 2H, J=8.02 Hz), 7.13 (d, 1H, J=16.19 Hz), 7.04 (d, 1H, J=7.98 Hz), 6.74 (s, 1H), 2.42 (s, 3H)

### Example 62

### 6-methyl-2-(4-methylaminostyryl)benzofuran trifluoroacetate (Compound 62)

¹H NMR (400 MHz, MeOD) δ 7.63 (d, 2H, J=8.59 Hz), 7.39 (d, 1H, J=7.92 Hz), 6.62 (d, 1H, J=6.62 Hz), 7.22 (d, 3H, J=8.89 Hz), 7.09 (d, 1H, J=16.2 Hz), 7.09 (d, 1H, J=7.96 Hz), 6.72 (s, 1H), 3.02 (s, 3H), 2.97 (s, 3H)

### Example 63

### 6-methyl-2-(4-diethylaminostyryl)benzofuran (Compound 63)

¹H NMR (400 MHz, CDCl₃) δ 7.40 (d, 2H, J=8.30 Hz), 7.36 (d, 1H, J=8.10 Hz), 7.25 (s, 1H), 7.20 (d, 1H, J=16.1 Hz), 7.01 (d, 1H, J=7.32 Hz), 6.76 (d, 1H, J=7.32 Hz), 6.76 (d, 1H, J=16.05 Hz) 6.67 (d, 1H, J=8.20 Hz), 6.50 (s, 1H), 3.39 (d, 4H, J=6.81 Hz), 2.47 (s, 3H), 1.29-1.26 (m, 6H)

### Example 64

### 6-methyl-2-(4-methoxystyryl)benzofuran (Compound 64)

¹H NMR (300 MHz, CDCl₃) δ 7.48 (d, 2H, J=8.73 Hz), 7.40 (d, 1H, J=7.88 Hz), 7.27-7.21 (m, 2H), 7.03 (d, 1H, J=7.96 Hz), 6.92 (d, 2H, J=8.6 Hz), 6.86 (d, 1H, J=16.15 Hz), 6.59 (s, 1H), 3.88 (s, 3H), 2.48 (s, 3H)

### Example 65

### 6-methyl-2-(3,4-dimethoxystyryl)benzofuran (Compound 65)

¹H NMR (400 MHz, CDCl₃) δ 7.35 (d, 1H, J=7.84 Hz), 7.30-7.22 (m, 3H), 7.09-7.07 (m, 3H), 6.86-6.84 (m, 2H) 6.57 (s, 1H), 3.93 (d, 6H, J=14.2 Hz), 2.44 (s, 3H)

### Example 66

### 6-(2-(2-(2-fluoroethoxy)ethoxy)ethoxy)-2-(4-methylaminostyryl)benzofuran (Compound 66)

¹H NMR (400 MHz, CDCl₃) δ 7.36 (d, J=8.8 Hz, 1H), 7.34 (d, J=8.8 Hz, 1H), 7.31 (d, J=8.8 Hz, 1H), 7.15 (d, J=16 Hz, 1H), 7.02 (d, J=1.6 Hz, 1H), 6.84 (dd, J=2.4, 8.4 Hz, 1H), 6.75 (d, J=16 Hz, 1H), 6.60 (d, J=8.8 Hz, 2H), 6.48 (s, 1H), 4.57 (dt, J=47.6, 4.0 Hz, 2H), 4.19 (t, J=4.8, 2H), 3.90 (t, J=4.8 Hz, 2H), 3.80 (m, 6H), 2.87 (s, 3H).

### Example 67

### 6-hydroxy-2-(4-aminostyryl)benzofuran (Compound 67)

¹H NMR (400 MHz, CDCl₃) δ 7.36 (d, J=8.4 Hz, 1H), 7.29 (d, J=8.4 Hz, 1H), 7.14 (d, J=16 Hz, 1H), 6.91 (d, J=32 Hz, 1H), 6.76 (d, J=16 Hz, 1H), 6.72 (d, J=8.8 Hz, 1H), 6.67 (dd, J=5.2, 8.4 Hz, 2H), 6.54 (d, J=36 Hz, 1H), 6.38 (dd, J=13, 92 Hz, 1H), 4.84 (br s, OH), 3.79 (br s, NH₂).

### Example 68

### 6-hydroxy-2-(4-methylaminostyryl)benzofuran (Compound 68)

¹H NMR (400 MHz, DMSO-d₆) δ 9.52 (s, NHMe), 7.35 (d, Hz, 1H), 7.31 (d, J=8.4 Hz, 1H), 7.00 (d, J=16 Hz, 1H), 6.87 (s, 1H), 6.84 (d, J=16 Hz, 1H), 6.69 (dd, J=2.0, 8.0 Hz, 1H), 6.62 (s, 1H), 6.53 (d, J=8.8 Hz, 2H), 5.98 (d, J=4.8 Hz, 1H), 2.70 (d, J=4.8 Hz, 3H).

### Example 69

### 6-hydroxy-2-(4-diethylaminostyryl)benzofuran (Compound 69)

¹H NMR (400 MHz, CDCl₃) δ 7.38 (d, J=8.8 Hz, 2H), 7.35 (d, J=8.4 Hz, 1H), 7.15 (d, J=16 Hz, 1H), 7.01 (d, J=2.4 Hz, 1H), 6.82 (dd, J=2.4, 8.4 Hz, 1H), 6.74 (d, J=16 Hz, 1H), 6.66 (d, J=8.8 Hz, 2H), 6.47 (s, 1H), 4.75 (s, OH), 3.39 (q, J=6.8 Hz, 4H), 1.19 (t, J=7.2 Hz, 6H).

### Example 70

### 5,6-dimethoxy-2-(4-methylaminostyryl)benzofuran (Compound 70)

¹H NMR (400 MHz, CDCl₃) δ 7.71 (d, J=7.71 Hz, 1H), 7.19 (m, 4H), 6.85 (d, J=1.84 Hz, 2H), 6.61 (d, J=7.8 Hz, 2H), 6.30 (d, J=10.44 Hz, 1H), 3.96 (s, 3H), 3.94 (s, 3H), 3.93 (s, 3H)

### Experimental Example 1

### Inhibitory Effect on the Formation of Beta-Amyloid Fibrils In Vitro (Tht Fluorescence Assay)

In order to investigate the inhibitory effect on the formation of beta-amyloid fibrils, the inventive compounds were examined as follows:

In this experiment, between the two types of beta-amyloid proteins, i.e., beta-amyloid 40 and beta-amyloid 42, beta-amyloid 42 was employed, which is a major target for the development of a therapeutic drug due to its strong neurotoxicity (Hammarstrom, P. et al., Science 2003, 299, 713; and Cai, X. D. et al., Science 1993, 259, 514).

Beta-amyloid 42 (Aβ42) was dissolved in dimethylsulfoxide (DMSO) to form a 250 mM Aβ42 stock solution. In addition, ThT (thioflavin T) was dissolved in distilled water to a concentration of 1 mM and subsequently diluted with 50 mM glycin buffer (pH 8.5) to yield a 5 μM ThT stock solution.

45 μL of PBS (phosphate buffer saline, pH 7.4) was added to each of a 96 well fluorescence microplate (white, F-bottom). 5 μL of the 250 μM Aβ42 stock solution was added to each well. The final concentration of each compound obtained in Examples was in a range of 10 to 0.001 μM by adding 2 μL of a solution, which is prepared by dissolving the subject compounds obtained in Examples in DMSO, to each well. At this time, the final concentration of Aβ42 in each well was 25 μM. The plate was then incubated at room temperature for 1 hr, and 150 μL of the 5 μM ThT stock solution was added to each well.

The fluorescence intensity of each well was determined with the multi label fluorescence counter (LS-55 Luminescence spectrometer, Perkin Elmer) at an excitation wavelength of 450 nm (excitation slit width: 10 nm) and an emission wavelength of 482 nm (emission slit width: 10 nm), while adjusting counting time to 1 second. The control group was prepared by adding PBS solution, Aβ42 and DMSO, without adding the inventive compound. % Inhibition on the formation of beta-amyloid fibrils was calculated in accordance with the following equation and IC_{5O} was calculated by using GraphPad Prism version 4.03 Program.

% Inhibition=[1−(C−D)/(A−B)]×100 Equation 1%

A (control group): fluorescence intensity in a group treated with PBS solution, Aβ42, and DMSO

B (blank): fluorescence intensity in a group treated with PBS solution and DMSO

C (experimental group): fluorescence intensity in a group treated with PBS solution, Aβ42, DMSO, and the inventive compound

D (compensation value to the experimental group): fluorescence intensity in a group treated with PBS solution, DMSO, and the inventive compound

% Inhibition and IC_{5O} value for the inhibitory effect on the formation of Aβ42 at 10 μM, compared with those of the comparative compounds were shown in Tables 1 to 7 below.

As comparative compounds, curcumin (Sigma) known as a material having a potent inhibitory effect against Aβ42 formation, 2-[2-(2-(dimethylaminothiazol-5-yl)ethenyl]benzothiazole (disclosed in EP 1655287, Comparative Example 1) and 2-(4-dimethylaminophenylethenyl)benzothiazole (disclosed in WO02/16333, Comparative Example 2) were employed.

Tables 1 to 7 below represent the results of the experiments performed separately. Therefore, the IC₅₀ values of curcumin in Tables 1 to 7 may vary depending on the degree of beta-amyloid 42 accumulation or the state of ThT. The inhibitory effect on the beta-amyloid 42 formation of the inventive compound can be evaluated by relatively comparing the IC₅₀ value with those of comparative compounds shown in each Table.

TABLE 1

| Compound | R¹ | R² | R³ | R⁴ | IC₅₀^{a} (μM) |
|---|---|---|---|---|---|
| 1 | H | H | N(CH₃)₂ | H | 2.300 |
| 2 | OCH₃ | H | N(CH₃)₂ | H | 0.810 |
| 3 | OH | H | N(CH₃)₂ | H | 0.163 |
| 4 | CH₃ | H | N(CH₃)₂ | H | 0.128 |
| 5 | F | H | N(CH₃)₂ | H | 1.980 |
| 6 | Cl | H | N(CH₃)₂ | H | 0.199 |
| 7 | Br | H | N(CH₃)₂ | H | 0.470 |
| 8 | I | H | N(CH₃)₂ | H | 0.168 |
| 9 | H | OCH₃ | N(CH₃)₂ | H | 0.070 |
| 10 | H | OH | N(CH₃)₂ | H | 3.619 |
| 11 | H | CH₃ | N(CH₃)₂ | H | 3.440 |
| 12 | H | F | N(CH₃)₂ | H | 2.191 |
| 13 | H | Cl | N(CH₃)₂ | H | 3.206 |
| 14 | H | Br | N(CH₃)₂ | H | 1.341 |
| 15 | H | I | N(CH₃)₂ | H | 2.798 |
| 16 | OCH₃ | H | NH₂ | H | 0.820 |
| 17 | OCH₃ | H | NHCH₃ | H | 0.078 |
| 18 | OH | H | NH₂ | H | 2.950 |
| 19 | OH | H | NHCH₃ | H | 2.160 |
| 20 | H | OCH₃ | NH₂ | H | 5.440 |
| 21 | H | OCH₃ | NHCH₃ | H | 0.800 |
| 22 | OCH₃ | H | N(CH₃)₂ | OCH₃ | 0.156 |
| 23 | H | OCH₃ | N(CH₃)₂ | OCH₃ | 0.528 |
| curcumin | | | | | 0.800 |

^{a}In vitro ThT analysis (Aβ42: 25 μM)

TABLE 2

| | | | | | % | |
|---|---|---|---|---|---|---|
| Com- | | | | | Inhibition | IC₅₀^{a} |
| pound | R¹ | R² | R³ | R⁴ | (10 μM) | (μM) |
| 33 | Cl | H | OCH₃ | H | 51.81 | >100 |
| 34 | Cl | H | OCH₃ | OCH₃ | 72.49 | 0.181 |
| 35 | OCH₃ | H | N(CH₂CH₃)₂ | H | 55.06 | 1.181 |
| 37 | OCH₃ | H | OCH₃ | H | 67.89 | 0.707 |
| 38 | OCH₃ | H | OCH₃ | OCH₃ | 81.10 | 1.562 |
| 42 | CH₃ | H | OCH₃ | H | 69.73 | 15.90 |
| 43 | CH₃ | H | OCH₃ | OCH₃ | 69.43 | 10.52 |
| 56 | OH | H | N(CH₂CH₃)₂ | H | 81.64 | 1.301 |
| 63 | H | CH₃ | OCH₃ | H | 76.96 | 3.537 |
| curcumin | | | | | 92.14 | 1.313 |

^{a}In vitro ThT analysis (Aβ42: 25 μM)

TABLE 3

| | | | | | % Inhibition | |
|---|---|---|---|---|---|---|
| Compound | R¹ | R² | R³ | R⁴ | (10 μM) | IC₅₀^{a} (μM) |
| 24 | H | H | NH₂ | H | 70.70 | — |
| 25 | H | H | NHCH₃ | H | 59.02 | — |
| 26 | H | H | N(CH₂CH₃)₂ | H | 4.54 | — |
| 27 | H | H | OCH₃ | H | 58.30 | — |
| 39 | CH₃ | H | NH₂CF₃COOH | H | 60.23 | — |
| 40 | CH₃ | H | NHCH₃CF₃COOH | H | 70.17 | — |
| 41 | CH₃ | H | N(CH₂CH₃)₂ | H | <1.00 | — |
| 57 | H | OCH₃ | N(CH₂CH₃)₂ | H | 62.79 | — |
| 58 | H | OCH₃ | OCH₃ | H | 57.16 | — |
| 60 | H | OCH₃ | NHCH₃ | OCH₃ | N.D. | — |
| 61 | H | CH₃ | NH₂CF₃COOH | H | 57.54 | — |
| 62 | H | CH₃ | NHCH₃CF₃COOH | H | 46.55 | — |
| 64 | H | CH₃ | N(CH₂CH₃)₂ | H | 33.31 | — |
| curcumin | | | | | 78.31 | 2.964 |

^{a}In vitro ThT analysis (Aβ42: 25 μM)

TABLE 4

| | | | | | % Inhibition | |
|---|---|---|---|---|---|---|
| Compound | R¹ | R² | R³ | R⁴ | (10 μM) | IC₅₀^{a} (μM) |
| 29 | Cl | H | NH₂ | H | 82.83 | 1.985 |
| 31 | Cl | H | N(CH₂CH₃)₂ | H | 37.86 | 0.963 |
| 49 | I | H | OCH₃ | H | 38.01 | 16.80 |
| 50 | I | H | OCH₃ | OCH₃ | 62.43 | 5.793 |
| curcumin | | | | | 79.65 | 0.525 |

^{a}In vitro ThT analysis (Aβ42: 25 μM)

TABLE 5

| | | | | | % Inhibition | IC₅₀^{a} |
|---|---|---|---|---|---|---|
| Compound | R¹ | R² | R³ | R⁴ | (10 μM) | (μM) |
| 28 | H | H | OCH₃ | OCH₃ | 67.38 | 3.291 |
| 32 | Cl | H | NHCH₃ | OCH₃ | 47.61 | |
| 36 | OCH₃ | | NHCH₃ | OCH₃ | 24.49 | |
| 45 | (OCH₂CH₂)₃F | H | NHCH₃ | H | 53.46 | |
| 47 | I | H | N(CH₂CH₃)₂ | H | 63.32 | 1.053 |
| 51 | OCH₃ | OCH₃ | N(CH₃)₂ | H | 29.16 | |
| 52 | OCH₃ | OCH₃ | N(CH₂CH₃)₂ | H | 42.28 | |
| 53 | OCH₃ | OCH₃ | NHCH₃ | OCH₃ | 56.19 | |
| 55 | OCH₃ | OCH₃ | OCH₃ | OCH₃ | 51.37 | |
| 65 | H | CH₃ | OCH₃ | OCH₃ | 67.50 | 0.884 |
| 66 | H | (OCH₂CH₂)₃F | NHCH₃ | H | 61.69 | |
| 67 | H | OH | NH₂ | H | 60.24 | |
| 68 | H | OH | NHCH₃ | H | 61.93 | |
| 69 | H | OH | N(CH₂CH₃)₂ | H | 42.23 | |
| 70 | OCH₃ | OCH₃ | NHCH₃ | H | 25.76 | |
| curcumin | | | | | 67.84 | 1.599 |

^{a}In vitro ThT analysis (Aβ42: 25 μM)

TABLE 6

| | | | | | % Inhibition | |
|---|---|---|---|---|---|---|
| Compound | R¹ | R² | R³ | R⁴ | (10 μM) | IC₅₀^{a} (μM) |
| hydrochloride | H | OCH₃ | N(CH₃)₂HCl | H | 62.88 | 1.884 |
| of Ex. 9 | | | | | | |
| hydrochloride | OCH₃ | H | NHCH₃HCl | H | 74.14 | 0.661 |
| of Ex. 17 | | | | | | |
| 30 | Cl | H | NHCH₃ | H | 68.44 | 2.169 |
| 40 | CH₃ | | NHCH₃CF₃COOH | H | 73.47 | 5.047 |
| 44 | (OCH₂CH₂)₂F | H | NHCH₃ | H | 84.71 | 1.494 |
| 46 | I | H | NHCH₃ | H | 60.99 | 1.768 |
| 48 | I | H | NHCH₃ | OCH₃ | 74.19 | 1.047 |
| 54 | OCH₃ | OCH₃ | OCH₃ | H | 77.75 | 0.903 |
| 59 | H | OCH₃ | OCH₃ | OCH₃ | 79.90 | 1.023 |
| curcumin | | | | | 98.11 | 2.357 |

^{a}In vitro ThT analysis (Aβ42: 25 μM)

TABLE 7

| | | | | | % Inhibition | |
|---|---|---|---|---|---|---|
| Compound | R¹ | R² | R³ | R⁴ | (10 μM) | IC₅₀^{a} (μM) |
| 9 | H | OCH₃ | N(CH₃)₂ | H | 78.73 | 0.972 |
| 17 | OCH₃ | H | NHCH₃ | H | 74.61 | 0.725 |

| | | | |
|---|---|---|---|
| Comparative Example 1^{b} | 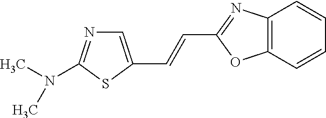 | 63.43 | 3.401 |
| Comparative Example 2^{c} | 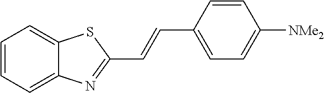 | 25.69 | 31.80 |
| curcumin | | 76.40 | 1.615 |

^{a} In vitro ThT analysis (Aβ42: 25 μM)
^{b}2-[2-(2-(dimethylaminothiazol-5-yl)ethenyl]benzothiazole disclosed in EP 1655287
^{c}2-(4-dimethylaminophenylethenyl)benzothiazole disclosed in WO02/16333

As can be seen from Tables 1 to 6, most of the inventive compounds, e.g., compound 3, 4, 6, 7, 8, 9 and its hydrochloride, 17 and its hydrochloride, 22, 23, 30, 34, 37, 44, 46, 47, 48, 54, 56, 59 and 65, showed superior % inhibition against the beta-amyloid 42 formation, to the comparative material, curcumin.

As can be seen from Table 7, the inventive compounds 9 and 17 showed superior % inhibition against the beta-amyloid 42 formation and superior IC₅₀ values to the compounds of Comparative Example 1 and 2 of the prior art.

### Experimental Example 2

### Pharmacokinetics and Passage Test Through the Blood-Brain Barrier in Mice and Rats

1. Pharmacokinetics Test

1) Test Animal and Administration of Test Compound

Three 7 week-old ICR mice (weight: about 30 g) and three 8 week-old SD rats (weight: about 250 g) were used per test group. A solution prepared by dissolving the compounds of Examples 9 and 17 in excipient (DMSO/Tween 20/saline: 0.1/0.6/2.3, v/v/v) were administered to each experimental animal. The test compound was orally administered in an amount of 10 mL per kg of body weight or intravenously administered in an amount of 5 mL per kg of body weight through the fine vein.

2) Blood Concentration Test

At 30 min, 1 hr, 2 hr, 4 hr, 10 hr and 24 hr after oral administration of the test compounds, the blood was collected from periorbital veins into the tube containing heparin (1000 IU/mL, 3 μl) for the mice, and from jugular vein for rats. The blood sample was centrifuged at 12,000 rpm for 2 min to obtain plasma. The obtained plasma was kept in freezer at −80° C. until the analysis.

3) Sample Analysis

The sample was analyzed using LC/MSMS system under the following condition:

| LC system | Agilent ® 1200series (Agilent Co.) | | |
|---|---|---|---|
| Moblie phase | Acetonitrile/0.1% 10 mM ammonium formate | | |
| | containing trifluoroacetic acid = 95/5 (v/v %) | | |
| Column | Luna ® phenyl hexyl (2.0 × 50 mm, | | |
| | 5 μm, Phenomenex Co.) | | |
| Flow | 0.2 mL/min | | |
| MS system | API ® 5000 (Applied Biosystems/MDS | | |
| | SCIEX Co.) | | |
| Ionization mode | Turbo Ion spray Ionization mode (positive) | | |
| Curtain gas (CUR) | 20 psi | Collision gas (CAD) | 6 psi |
| Ion voltage | 5400 V | GS 1 // GS 2 | 50/50 psi |
| Turbo gas temperature | 480° C. | CUR, CAD, GS(1, 2) | nitrogen |

MRM (multi reaction monitoring) parameter

| | | | | |
|---|---|---|---|---|
| compound | Q1/Q3 | DP | CE | CXP |
| Test compound | 294.317/264.2 | 71 | 23 | 30 |

* Q1: precursor ion (m/z)
Q3: product ion (m/z)

The sample was pretreated as follows:

50 μL of plasma was placed in 2.0 mL of tube having a cap (Eppendorf Co.) and acidified by adding 20 μL of 0.1% formic acid thereto. An internal standard solution and 1 mL of ethyl acetate as an extract solvent were added to the resultant solution. The resultant solution was mixed using thermomixer (Eppendorf Co.) at 1400 rpm for 5 min, and then subjected to centrifuge (Eppendorf Co.). The supernatant was collected and concentrated at 35° C. using cyclone. The residue was re-dissolved in 50 μL of moblie phase and 5 μL of the resulting solution was injected into LC/MS and analyzed.

2. Passage Test Through the Blood-Brain Barrier

1) Test Animal and Administration of Test Compound

Three 7 week-old ICR mice (weight: about 30 g) and three 8 week-old SD rats (weight: about 250 g) were used per test group. A solution prepared by dissolving the compounds of Examples 9 and 17 in excipient (DMSO/Tween 20/saline: 0.1/0.6/2.3, v/v/v) were administered to each experimental animal. The test compound was orally administered in an amount of 10 mL per kg of body weight or intravenously administered in an amount of 5 mL per kg of body weight through the fine vein.

2) Measurement of Concentration in Blood and Tissue (Simultaneous Test)

(1) Blood-Sampling

At 30 min, 1 hr, 2 hr, 4 hr, 10 hr and 24 hr after administration of test compound, the mice and rats were subjected to insufflations narcosis using isoflorane, followed by cutting the abdomen open. 1 mL of blood was collected from abdominal veins into the tube containing heparin (1000 IU/mL, 3 μl). The obtained blood sample was centrifuged at 12,000 rpm, for 2 min to obtain plasma. The obtained plasma was kept in freezer at −80° C. until the analysis.

(2) Organ Tissue-Sampling

The mice and rats from which the blood sample was obtained were subjected to bloodletting, and then, brain tissue of the mice and rats was collected. The brain tissue thus obtained was washed with physiological saline once or twice to remove blood. The weight of the brain tissue was measured after the removal of adipose tissue and peripheral tissue. 4% bovine serum albumin (BSA) solution diluted with 10-fold was added to the brain tissue. The resulting solution was subjected to homogenization using the homogenizer. The diluted homogenate thus obtained was placed in 2 ml of tube and was kept in freezer at −80° C. until the analysis. All of the treatments to the samples were performed in ice.

(3) Sample Analysis

The sample was analyzed using LC/MSMS system under the following condition:

| LC system | Agilent ® 1200series (Agilent Co.) | | |
|---|---|---|---|
| Moblie phase | Acetonitrile/0.1% 10 mM ammonium formate | | |
| | containing trifluoroacetic acid = 95/5 (v/v %) | | |
| Column | Luna ® phenyl hexyl (2.0 × 50 mm, | | |
| | 5 μm, Phenomenex Co.) | | |
| Flow | 0.2 mL/min | | |
| MS system | API ® 5000 (Applied Biosystems/MDS | | |
| | SCIEX Co.) | | |
| Ionization mode | Turbo Ion spray Ionization mode (positive) | | |
| Curtain gas (CUR) | 20 psi | Collision gas (CAD) | 6 psi |
| Ion voltage | 5400 V | GS 1 // GS 2 | 50/50 psi |
| Turbo gas temperature | 480° C. | CUR, CAD, GS(1, 2) | nitrogen |

MRM (multi reaction monitoring) parameter

| | | | | |
|---|---|---|---|---|
| compound | Q1/Q3 | DP | CE | CXP |
| Test compound | 294.317/264.2 | 71 | 23 | 30 |

* Q1: precursor ion (m/z)
Q3: product ion (m/z)

The sample was pretreated as follows:

50 μL of plasma were placed in 2.0 mL of tube having a cap (Eppendorf Co.) and acidified by adding 20 μL of 0.1% formic acid thereto. An internal standard solution and 1 mL of ethyl acetate as an extract solvent were added to the resultant solution. The resultant solution was mixed using thermomixer (Eppendorf Co.) at 1400 rpm for 5 min and then subjected to centrifuge (Eppendorf Co.). The supernatant was collected and concentrated at 35° C. using cyclone. The residue was re-dissolved in 50 μL of moblie phase and 5 μL of the resulting solution was injected into LC/MS and analyzed.

3. Results

Table 8 shows the results of pharmacokinetics and passage test through the blood-brain barrier in mice of the compounds of Examples 9 and 17, and Table 9 shows the results in rats.

In Tables 8 and 9, “iv” refers to a intravenous injection; “po” refers to a per oral; “AUC plasma” refers to an area under the plasma level-time curve; “Cmax” refers to a maximum plasma concentration; “Tmax” refers to a time to reach Cmax, “BA” refers to a bioavailability (%) according to Equation 2 below; “AUC Brain” refers to a area under the brain tissue level-time curve; and “AUCBrain/AUCPlasma” refers to a passage rate of the test compound to the brain.

Bioavailability (%)=[(AUCpo/AUCiv)×(Doseiv/Dosepo)×100] Equation 2

wherein, AUCpo means an area under the blood concentration time curve (AUC) after the per oral administration, AUCiv means an AUC after the intravenous injection, Doseiv means an amount of the intravenous injection, and Dosepo means an amount of the per oral administration.

TABLE 8

| | Compound of | | Compound of | |
|---|---|---|---|---|
| | Example 9 (n = 3) | | Example 17 (n = 3) | |
| | 1.25 mg/ | | 1.25 mg/ | |
| Parameter | kg, iv | 2.5 mg/kg, po | kg, iv | 2.5 mg/kg, po |
| AUC Plasma | 503.35 | 645.95 | 237.04 | 325.95 |
| (ng · h/ml) | | | | |
| Cmax (ng/ml) | | 172.0 | | 96.53 |
| Tmax (hr) | | 0.83 | | 0.83 |
| BA (%) | 64.2 | | 68.8 | |
| AUC Brain | 6101.86 | 8137.42 | 2525.45 | 2354.58 |
| (ng · h/ml) | | | | |
| Cmax (ng/ml) | | 899.33 | | 462.67 |
| Tmax (hr) | | 4.0 | | 2.0 |
| AUCBrain/ | 1210 | 1260 | 1060 | 720 |
| AUC Plasma | | | | |
| (%) | | | | |

TABLE 9

| | Compound of | | Compound of | |
|---|---|---|---|---|
| | Example 9 (n = 3) | | Example 17 (n = 3) | |
| | 1.25 mg/ | | 1.25 mg/ | |
| | kg, iv | 2.5 mg/kg, po | kg, iv | 2.5 mg/kg, po |
| AUC Plasma | 1637.46 | 908.50 | 1144.47 | 516.05 |
| (ng · h/ml) | | | | |
| Cmax (ng/ml) | | 215.0 | | 122.00 |
| Tmax (hr) | | 1.67 | | 1.33 |
| BA (%) | 27.8 | | 22.4 | |
| AUC Brain | 17939.63 | 15241.92 | 8740.33 | 11237.67 |
| (ng · h/ml) | | | | |
| Cmax (ng/ml) | | 1309.00 | | 1406.67 |
| Tmax (hr) | | 2.0 | | 1.67 |
| AUCBrain/ | 1100 | 1680 | 760 | 2180 |
| AUC Plasma | | | | |
| (%) | | | | |

As can be seen from Tables 8 and 9, the compounds of Examples 9 and 17 showed a high degree of AUC which is suitable for a therapeutic agent for brain disease and superior bioavailability.

Also, from the result of passage test through the blood-brain barrier, it is found that the compounds of Examples 9 and 17 showed 100% or more of passage ability compared with plasma, which is suitable for a therapeutic agent for brain disease.

### Experimental Example 3

### Inhibitory Effect on hERG Potassium Ion Channel

1) Model Cell Line and Culture

A HEK-hERG cell line (lonGate Biosciences, Frankfurt, Germany Co.), which expresses hERG stably, was cultured in a DMEM (Dulbecco's Modified Eagle's Medium, Sigma Co., St. Louis, Mo., USA) supplemented with 10% fetal bovine serum (FBS, Cambrex, Walkersville, Md., USA) and 0.5 mg/mL zeocin (Invitrogen, Carlsbad, Calif., USA). The cell line was subcultured 5 days after culture when 80% confluency was reached.

2) Preparation of Test Solution and Test Drug

(1) Test Solution

A solution within an electrode used to measure the potassium ion current is composed of 115 mM K-aspartate, 20 mM KCl, 10 mM EGTA, 10 mM HEPES, 2.5 mM tris-phosphocreatine, 0.1 mM Na₂GTP and 5 mM MgCl₂ (pH 7.2, 290 mOsm/Kg H₂O). A solution for an extracellular perfusate is composed of 135 mM NaCl, 5 mM KCl, 1 mM MgCl₂, 2 mM CaCl₂, 10 mM glucose, and 10 mM HEPES (pH 7.2, 300 mOsm/Kg H₂O).

(2) Test Drug

Test drug solutions were prepared by respectively diluting the inventive compounds with extracellular perfusate to a desired concentration. The prepared test drug solution was placed in a 7-array polyethylene tube connected into a capillary column for gas chromatography and was dropped from the tip of the column at a height of 100 μm or less to the HEK-hERG cell line.

3) Measurement of Ion Current

Potassium ion current was measured using EPC10 (Instrutech Co., NY, USA) patch clamp amplifier in accordance with the conventional whole-cell patch clamp method. Electrode used in the measurement was a borosilicate glass capillary (external diameter: 1.65 mm, inside diameter: 1.2 mm, Corning 7052, Gamer Glass Co., Claremont, Calif., USA) prepared by using P-97 Flaming-Brown micropipette puller (Sutter Instrument Co.). The electrode was coated with Sylgard 184 (Dow Corning Co., Midland, Mich., USA) and trimmed with microforge (Narishige Co., Tokyo, Japan). The electrode had a resistance of 2-3MΩ when filled with a solution. A culture dish containing HEK-hERG cells was placed in an inverted microscope (Nikon Co.) and extracellular perfusate containing the inventive compound was perfused at a rate of 1-2 mL/min. The membrane capacitance and series resistance of cell membrane were calibrated by 80% or more and potassium ion current was measured at a sampling rate of 2 kHz and a low-pass filter of 2 kHz (−3 dB; 8-pole Bassel filter). The test is conducted at room temperature (21-24° C.).

4) Data analysis and Statistics

The results were analyzed using Pulse/Pulsefit (v9.0, HEKA Elcktronik, Lambrecht, Germany) and Igor macro. The results were given as meant standard error. IC₅₀ of a test compound, the concentration of the test compound at which 50% ion current was inhibited, was obtained from a concentration-response curve by using the Hill equation [Block=(1+IC₅₀/[drug]ⁿ)⁻¹]. The following Table 10 shows the results.

TABLE 10

| Test Compound | Inhibitory efficacy (IC₅₀) |
|---|---|
| Compound 9 | 52.14 ± 3.68 μM |
| Compound 17 | 47.90 ± 5.26 μM |

As can be seen from Table 10, the inventive compounds of Examples 9 and 17 showed an insignificant inhibitory efficacy on hERG potassium ion channel and accordingly, they are considered to be non-cardiotoxic.

### Experimental Example 4

### In Vivo Fear Conditioning Test

1) Transgenic Mice

The tails of 3-week old mice born from Male B6C3-Tg (APPswe, PSEN1 dE9, Jackson Laboratory Co., Maine, A) and female B6C3F1 (central Lab. Animal Inc., Korea) were cut in lengths of about 0.5 cm (tail biopsy). The genomic DNAs were extracted from the tail samples and subjected to a genotype analysis to screen transgenic double tg mice.

Such transgenic mice are generally used in a dementia treating efficacy test, because they when above 5 months old, exhibit a phenotype identical to that of a human dementia patient due to the accumulation of the beta-amyloid in the brain.

2) Drug Administration

From 5 months to 12 months after the birth, the mice were administered orally with the compound of Example 9, in an amount of 30 mg/kg or 100 mg/kg every day. As a comparative compound, tramiprosate (Aisen, P. S. et. al., Curr. Alzheimer Res. 2007, 4, 473) was used, which binds to beta-amyloid protein to inhibit the deposition and cytotoxicity to the protein. The compounds were administrated.

3) Fear Conditioning Test

At the First day of training, the mice were placed in a conditioning box and adjusted for 2 min. The fear conditioning was performed with a conditional stimulus (CS) of 75 dB for 20 sec, together with an electrical stimulus (unconditional stimulus (US)) of 0.5 mA for final 2 sec in the conditional stimulus period. After 1 min, the animals were transferred to a cage. After 24 hrs, the retention test was performed. The animals were placed in the same conditioning box as used above and observed for 5 min. The freezing response was measured without CS and US. The freezing response is defined in the state of the animals keeping still except for breathing.

4) Measurement of Beta-Amyloid Content

After the fear conditioning test, the brain was extracted and was subject to the histochemical staining. The amount of beta-amyloid deposited in the brain was measured using ELISA method. The results are shown in the following Table 11.

TABLE 11

| Test compound | Dose (mg/kg) | Increase rate (fold)^{a} |
|---|---|---|
| Compound 9 | 30 | 3.2 |
| | 100 | 3.8 |
| Tramiprosate | 30 | 2.3 |
| | 100 | 1.8 |

^{a}increase rate based on the freezing (%) of Tg group (the control group treated with vehicle only).

As can be seen from Table 11, the inventive compound of Example 9 showed a much higher degree of memory in a dose-dependent manner, as compared with tramiprosate.

### Experimental Example 5

### Immunohistochemistry

The transgenic mice were screened and drug was administered thereto as described in 1) and 2) of Experimental Example 4. The brain was separated from the transgenic mouse and fixed in 10% neutral formalin solution. A region of the brain including the hippocampus and the cortex were subjected to removal, washing, dehydration and paraffin infiltration to obtain paraffin block including the brain tissue. The paraffin block was subjected to thin section in thickness of 8 μm to obtain the sections of all regions of hippocampus. Among them, 10 sections were elected at regular intervals. They were deparaffinized, hydrated, immersed in Mayer's hematoxylin for 1 min and rinsed with tap water. The rinsed tissue was reacted in an alkaline sodium chloride solution for 20 min, after then was reacted in an alkaline congo red solution for 20 min. The resulting tissue was washed with 100% ethyl alcohol, cleared with xylene and mounted using a synthetic mounting medium. In the hippocampus and cortex regions of the tissue preparations strained with congo red, congo red-positive beta-amyloid plagues were counted. The results are shown in the following Table 12.

TABLE 12

| | Concentration | Total reduction rate (%)^{a} |
|---|---|---|
| Test compound | (mg/kg) | (hippocampus, cortex) |
| Compound 9 | 30 | 41 (29, 43) |
| | 100 | 61 (64, 61) |
| Tramiprosate | 30 | 29 (7, 32) |
| | 100 | 11 (7, 12) |

^{a}reduction rate based on the freezing (%) of the Tg group

As can be seen from Table 12, the inventive compound of Example 9 showed a remarkable reduction rate of beta-amyloid deposition in a dose-dependent manner, as compared with tramiprosate.

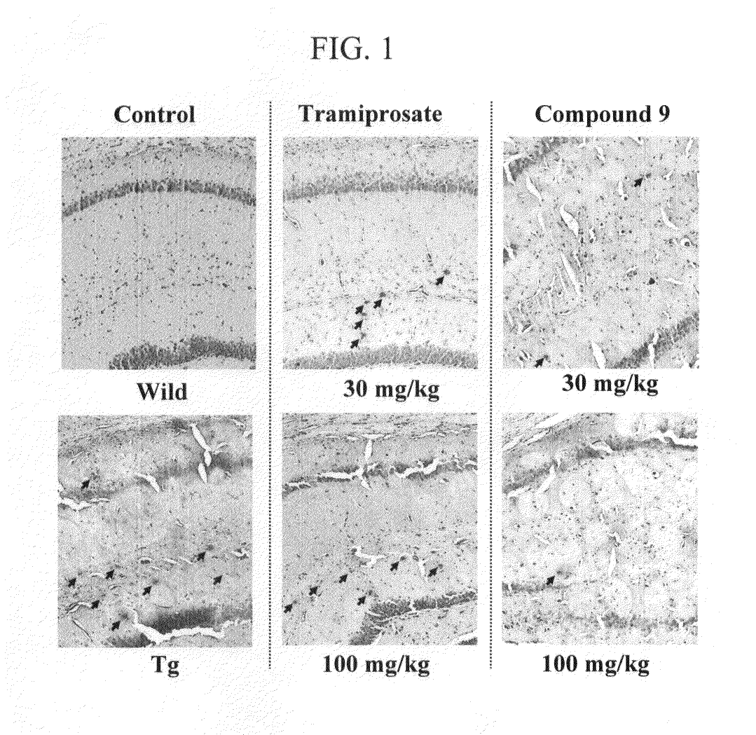

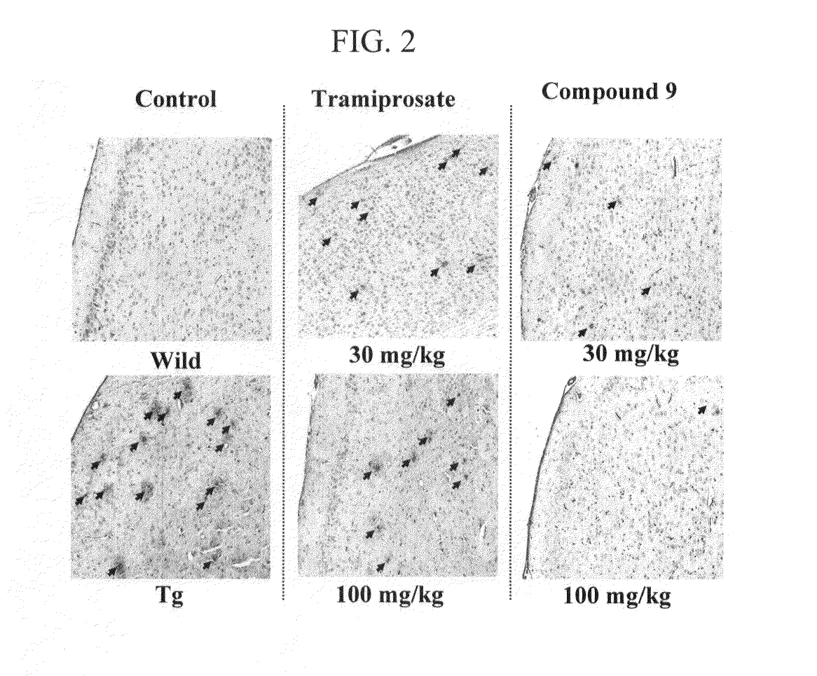

FIGS. 1 and 2 respectively show the hippocampus tissues and cortex tissues of the transgenic mice stained with the compound of Example 9 or the comparative compound, tramiprosate.

As can be seen from FIGS. 1 and 2, the inventive compounds of Example 9 showed a remarkably reduced beta-amyloid deposition as compared with tramiprosate.

### Experimental Example 6

### Biochemical Test

The transgenic mice were screened and drug was administered thereto as described in 1) and 2) of Experimental Example 4. The hippocampus tissue was extracted from transgenic mouse, placed in 8 fold amount of 5M guanidine HCl/50 mM Tris HCl, and subjected to homogenization using a homogenizer. The tissue homogenate thus obtained was allowed to stand for 3 hrs at room temperature, diluted by 50-folds with BSAT-DPBS (Dulbecco's phosphate buffered saline with 5% BSA and 0.03% Tween-20) including a protease inhibitor (Pierce®, Cat No. 78415). The content of beta-amyloid 42 was measured using Human beta-amyliod HS1-42 colorimetric kit (Invitrogen®, Cat No. #KHB3544). The results are shown in the following Table 13.

TABLE 13

| Test compound | Concentration (mg/kg) | Reduction rate (%)^{a} |
|---|---|---|
| Compound 9 | 30 | 41 |
| | 100 | 48 |
| Tramiprosate | 30 | 8 |
| | 100 | 16 |

^{a}reduction rate based on the content of beta amyloid 42 in the Tg group

As can be seen from Table 13, the inventive compound of Example 9 showed a remarkable reduction rate of beta-amyloid plague in a dose-dependent manner, as compared with tramiprosate.

While the invention has been described with respect to the above specific embodiments, it should be recognized that various modifications and changes may be made to the invention by those skilled in the art which also fall within the scope of the invention as defined by the appended claims.

1.-10. (canceled)

11. A compound of formula (I) or a pharmaceutically acceptable salt thereof: 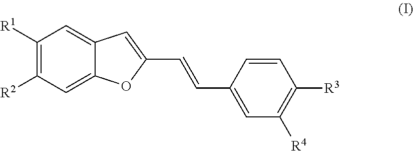
wherein,
R¹ and R² are each independently H, OH, halogen, C₁-C₃ alkoxy, C₁-C₃ alkyl, substituted poly(C₁-C₃ alkoxy) having one or more halogen or hydroxyl groups, or substituted pyranyl (C₁-C₃ alkoxy) having one or more C₁-C₃ alkyl groups, with the proviso that both R¹ and R² are not simultaneously H;
R³ is NH₂, C₁-C₃ alkylamino, C₁-C₃ dialkylamino, or C₁-C₃ alkoxy; and
R⁴ is H or C₁-C₃ alkoxy.

12. The compound of claim 11 or a pharmaceutically acceptable salt thereof, wherein
R¹ and R² are each independently H, OH, halogen, OCH₃, CH₃, (OCH₂CH₂)₂F, (OCH₂CH₂)₃F, or dimethylpyranylmethoxy, with the proviso that both R¹ and R² are not simultaneously H;
R³ is NH₂, NHCH₃, N(CH₃)₂, or OCH₃; and
R⁴ is H or OCH₃.

13. The compound of claim 11 or a pharmaceutically acceptable salt thereof, which is selected from the group consisting of:
5-methoxy-2-(4-dimethylaminostyryl)benzofuran;
5-hydroxy-2-(4-dimethylaminostyryl)benzofuran;
5-methyl-2-(4-dimethylaminostyryl)benzofuran;
5-fluoro-2-(4-dimethylaminostyryl)benzofuran;
5-chloro-2-(4-dimethylaminostyryl)benzofuran;
5-bromo-2-(4-dimethylaminostyryl)benzofuran;
5-iodo-2-(4-dimethylaminostyryl)benzofuran;
6-methoxy-2-(4-dimethylaminostyryl)benzofuran;
6-hydroxy-2-(4-dimethylaminostyryl)benzofuran;
6-methyl-2-(4-dimethylaminostyryl)benzofuran;
6-fluoro-2-(4-dimethylaminostyryl)benzofuran;
6-chloro-2-(4-dimethylaminostyryl)benzofuran;
6-bromo-2-(4-dimethylaminostyryl)benzofuran;
6-iodo-2-(4-dimethylaminostyryl)benzofuran;
5-methoxy-2-(4-aminostyryl)benzofuran;
5-methoxy-2-(4-methylaminostyryl)benzofuran;
5-hydroxy-2-(4-aminostyryl)benzofuran hydrochloride;
5-hydroxy-2-(4-methylaminostyryl)benzofuran hydrochloride;
6-methoxy-2-(4-aminostyryl)benzofuran;
6-methoxy-2-(4-methylaminostyryl)benzofuran;
5-methoxy-2-(3-methoxy-4-dimethylaminostyryl)benzofuran;
6-methoxy-2-(3-methoxy-4-dimethylaminostyryl)benzofuran;
5-chloro-2-(4-aminostyryl)benzofuran;
5-chloro-2-(4-methylaminostyryl)benzofuran;
5-chloro-2-(4-diethylaminostyryl)benzofuran;
5-chloro-2-(3-methoxy-4-methylaminostyryl)benzofuran;
5-chloro-2-(4-methoxystyryl)benzofuran;
5-chloro-2-(3,4-dimethoxystyryl)benzofuran;
5-methoxy-2-(4-diethylaminostyryl)benzofuran;
5-methoxy-2-(3-methoxy-4-methylaminostyryl)benzofuran;
5-methoxy-2-(4-methoxystyryl)benzofuran;
5-methoxy-2-(3,4-dimethoxystyryl)benzofuran;
5-methyl-2-(aminostyryl)benzofuran trifluoroacetate;
5-methyl-2-(4-methylaminostyryl)benzofuran trifluoro acetate;
5-methyl-2-(4-diethylaminostyryl)benzofuran;
5-methyl-2-(4-methoxystyryl)benzofuran;
5-methyl-2-(3,4-dimethoxystyryl)benzofuran;
5-(2-(2-fluoroethoxy)ethoxy)-2-(4-methylaminostyryl)benzofuran;
5-(2-(2-(2-fluoroethoxy)ethoxy)ethoxy)-2-(4-methylaminostyryl)benzofuran;
5-iodo-2-(4-methylaminostyryl)benzofuran;
5-iodo-2-(4-diethylaminostyryl)benzofuran;
5-iodo-2-(3-methoxy-4-methylaminostyryl)benzofuran;
5-iodo-2-(4-methoxystyryl)benzofuran;
5-iodo-2-(3,4-dimethoxystyryl)benzofuran;
5,6-dimethoxy-2-(4-dimethylaminostyryl)benzofuran;
5,6-dimethoxy-2-(4-diethylaminostyryl)benzofuran;
5,6-dimethoxy-2-(3-methoxy-4-methylaminostyryl)benzofuran;
5,6-dimethoxy-2-(4-methoxystyryl)benzofuran;
5,6-dimethoxy-2-(3,4-dimethoxystyryl)benzofuran;
5-hydroxy-2-(4-diethylaminostyryl)benzofuran;
6-methoxy-2-(4-diethylaminostyryl)benzofuran;
6-methoxy-2-(4-methoxystyryl)benzofuran;
6-methoxy-2-(3,4-dimethoxystyryl)benzofuran;
6-methoxy-2-(3-methoxy-4-methylaminostyryl)benzofuran;
6-methyl-2-(4-aminostyryl)benzofuran trifluoroacetate;
6-methyl-2-(4-methylaminostyrypbenzofuran trifluoroacetate;
6-methyl-2-(4-diethylaminostyryl)benzofuran;
6-methyl-2-(4-methoxystyryl)benzofuran;
6-methyl-2-(3,4-dimethoxystyryl)benzofuran;
6-(2-(2-(2-fluoroethoxy)ethoxy)ethoxy)-2-(4-methylaminostyryl)benzofuran;
6-hydroxy-2-(4-aminostyryl)benzofuran;
6-hydroxy-2-(4-methyl aminostyryl)benzofuran;
6-hydroxy-2-(4-diethylaminostyryl)benzofuran;
5,6-dimethoxy-2-(4-methylaminostyryl)benzofuran;
5-(2,2-dimethyltetrahydropyran-4-ylmethoxy)-2-(4-aminostryl)benzofuran;
5-(2,2-dimethyltetrahydropyran-4-ylmethoxy)-2-(4-methylaminostryl)benzofuran;
5-(2,2-dimethyltetrahydropyran-4-ylmethoxy)-2-(4-dimethylaminostryl)benzofuran;
6-(2,2-dimethyltetrahydropyran-4-ylmethoxy)-2-(4-aminostryl)benzofuran;
6-(2,2-dimethyltetrahydropyran-4-ylmethoxy)-2-(4-methylaminostryl)benzofuran; and
6-(2,2-dimethyltetrahydropyran-4-ylmethoxy)-2-(4-dimethylaminostryl)benzofuran.

14. A method for preparing the compound of formula (I) of claim 11, which comprises subjecting a compound of formula (II) to a Honer-Emmons reaction with a compound of formula (III) in an organic solvent in the presence of a base: 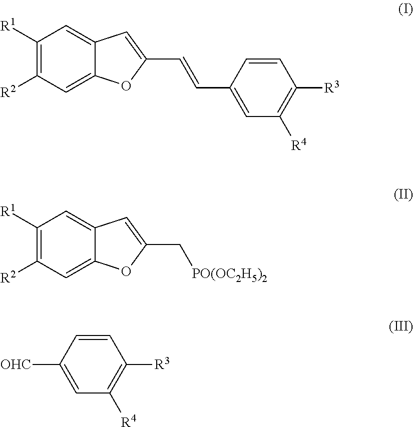
wherein,
R¹ and R² are each independently H, OH, halogen, C₁-C₃ alkoxy, C₁-C₃ alkyl, substituted poly(C₁-C₃ alkoxy) having one or more halogen or hydroxyl groups, or substituted pyranyl (C₁-C₃ alkoxy) having one or more C₁-C₃ alkyl groups, with the proviso that both R¹ and R² are not simultaneously H;
R³ is NH₂, C₁-C₃ alkylamino, C₁-C₃ dialkylamino, or C₁-C₃ alkoxy; and
R⁴ is H or C₁-C₃ alkoxy.

15. The method of claim 14, wherein the base is selected from the group consisting of an alkali metal hydride, an alkyl alkali metal compound, an alkali metal alkoxide, an alkali metal amide, and a mixture thereof.

16. The method of claim 14, wherein the organic solvent is ether.

17. The method of claim 14, which further comprises the step of subjecting the compound formed by the Honer-Emmons reaction to demethylation using a solution of boron trichloride, boron trifluoride, boron tribromide, or iodotrimethylsilane in the organic solvent.

18. A pharmaceutical composition for inhibiting the formation of beta-amyloid fibrils comprising the compound of formula (I) or its pharmaceutically acceptable salt of claim 11 as an active ingredient.

19. A pharmaceutical composition for preventing or treating a degenerative brain disease comprising the compound of formula (I) or its pharmaceutically acceptable salt of claim 11 as an active ingredient.

20. The pharmaceutical composition of claim 19, wherein the pharmaceutically acceptable salt is a salt of an acid selected from the group consisting of hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, toluenesulfonic acid, and a mixture thereof.

21. The method of claim 15, which further comprises the step of subjecting the compound formed by the Honer-Emmons reaction to demethylation using a solution of boron trichloride, boron trifluoride, boron tribromide, or iodotrimethylsilane in the organic solvent.

22. The method of claim 16, which further comprises the step of subjecting the compound formed by the Honer-Emmons reaction to demethylation using a solution of boron trichloride, boron trifluoride, boron tribromide, or iodotrimethylsilane in the organic solvent.
